(19) United States
(12) Patent Application Publication
(10) Pub. No.: US 2026/0341483 A1
(43) Pub. Date: Oct. 29, 2026

(54) SYSTEMS AND METHODS FOR A VALUE-BASED CARE MODEL BUILDER

(71) Applicant: Syntax Health, Inc., Atlanta, GA (US)

(72) Inventors: William Kelly HUDGINS, Seguin, TX (US); Emily Jo WALKER, Danville, PA (US); Rachael Nadesda McCalla JONES, Roswell, GA (US)

(73) Assignee: Syntax Health, Inc., Atlanta, GA (US)

(21) Appl. No.: 19/189,886

(22) Filed: Apr. 25, 2025

(51) Int. Cl. G16H 40/20

(52) U.S. Cl. CPC G16H 40/20

(57) ABSTRACT

This disclosure provides systems and methods for value-based care (VBC) contract construction. Systems for VBC contract construction may include a data manager implemented on a remote cloud service and configured to obtain, a first request from a client layer, the first request generated based on an action at a user device, and construct, in response to the first request, at least one VBC value using at least one of a plurality of VBC tools. Systems for VBC contract construction may include the client layer communicatively coupled to the data manager and configured to process the at least one VBC value to generate a user-interface for an implementation at the user device, the user-interface configured to enable a user to view VBC informatics based on the at least one VBC value on a display device and to make inputs to control visualization of the VBC informatics displayed to the user.

### BACKGROUND

Value-based care (VBC) contracting represents a paradigm shift in healthcare reimbursement models, emphasizing member outcomes and cost efficiency over the volume of services provided. While this approach aims to improve the quality of care and reduce healthcare costs, several inherent technical challenges can impact the effectiveness and implementation of VBC contracts. Accurate measurement and attribution of member outcomes are complicated by variability in member populations and differences in care delivery practices. Additionally, regulatory and compliance complexities add another layer of difficulty, as VBC contracts may navigate a landscape of evolving healthcare policies and regulations.

Although current techniques for value-based care contracting are based on advancements in healthcare delivery and reimbursement modelling, current VBC techniques may still be ineffective to achieve ideal results. Accordingly, there is an impetus to improve VBC techniques to overcome current challenges by implementing improvements including, for example:
enhancing the reliability of outcome measurement, increasing the accuracy of member attribution, reducing inefficiencies associated with data interoperability, and decreasing the cost of implementation. These improvements may be useful to address the technical challenges inherent in VBC contracting and to improve member care and cost efficiency.

Consequently, there exists a need for further improvements to value-based care contracting to overcome the aforementioned challenges.

### BRIEF SUMMARY

Various details of the present disclosure are hereinafter summarized to provide a basic understanding. This summary is not an exhaustive overview of the disclosure and is neither intended to identify certain elements of the disclosure, nor to delineate the scope thereof. Rather, the primary purpose of this summary is to present some concepts of the disclosure in a simplified form prior to the more detailed description that is presented hereinafter.

According to an aspect consistent with the present disclosure, a system for VBC contract construction may include a data manager implemented on a remote cloud service and configured to obtain, a first request from a client layer, the first request generated based on an action at a user device, and construct, in response to the first request, at least one VBC value using at least one of a plurality of VBC tools. A system may further include the client layer communicatively coupled to the data manager and configured to process the at least one VBC value to generate a user-interface for an implementation at the user device, the user-interface configured to enable a user to view VBC informatics based on the at least one VBC value on a display device and to make inputs to control visualization of the VBC informatics displayed to the user.

In another aspect, a system for VBC contract construction may include a user device communicatively coupled to a client layer over a network and configured to output a first request to the client layer based on user inputs, the first request configured to cause at least one of a plurality of VBC tools to generate at least one VBC value. A system may further include a user-interface displayed on the user device, the user-interface configured to enable a user to view VBC informatics based on the at least one VBC value displayed on the user device and to make the user inputs to control visualization of the VBC informatics displayed to the user.

Other aspects of the present disclosure provide:
one or more devices (e.g., apparatuses) operable, configured, or otherwise adapted to perform the aforementioned methods as well as those described elsewhere herein;
a non-transitory, computer-readable media including computer-executable instructions that, when executed by a processor of an apparatus, cause the apparatus to perform the aforementioned methods as well as those described elsewhere herein;
a computer program product embodied on a computer-readable storage medium comprising code for performing the aforementioned methods as well as those described elsewhere herein; and
an apparatus including means for performing the aforementioned methods as well as those described elsewhere herein. By way of example, an apparatus may include a processing system, or processing systems cooperating over one or more message passing interfaces.

Any combinations of the various aspects and implementations disclosed herein can be used in a further aspect, consistent with the disclosure. These and other aspects and features can be appreciated from the following description of certain aspects presented herein in accordance with the disclosure and the accompanying drawings and claims.

### BRIEF DESCRIPTION OF THE DRAWINGS

So that the manner in which the above recited features of the present disclosure can be understood in detail, a more particular description of the disclosure, briefly summarized above, may be had by reference to embodiments, some of which are illustrated in the appended drawings. It is to be noted, however, that the appended drawings illustrate only example embodiments and are therefore not to be considered limiting of its scope, and may admit to other equally effective embodiments.

FIG. 1 depicts a system for VBC contract construction, in accordance with one or more aspects of the present disclosure.

FIG. 2 depicts data flow through a system for VBC contract construction, in accordance with one or more aspects of the present disclosure.

FIG. 3 depicts data flow through a system for VBC contract construction, in accordance with one or more aspects of the present disclosure.

FIG. 4 depicts a VBC toolkit and a user experience (UX) associated therewith, in accordance with one or more aspects of the present disclosure.

FIG. 5 illustrates a contract manager screen displayed in accordance with VBC procedures , in accordance with one or more aspects of the present disclosure.

FIG. 6A illustrates an affinity review screen displayed in accordance with affinity tool procedures, in accordance with one or more aspects of the present disclosure.

FIG. 6B illustrates an affinity screen displayed in accordance with affinity tool procedures, in accordance with one or more aspects of the present disclosure.

FIG. 6C illustrates an affinity select screen displayed in accordance with affinity tool procedures, in accordance with one or more aspects of the present disclosure.

FIG. 7 illustrates an attribution screen displayed in accordance with attribution tool procedures, in accordance with one or more aspects of the present disclosure.

FIG. 8 illustrates a model type screen displayed in accordance with model type tool procedures, in accordance with one or more aspects of the present disclosure.

FIG. 9A-9B illustrate a cost term screen displayed in accordance with cost term tool procedures, in accordance with one or more aspects of the present disclosure.

FIG. 10 illustrates a quality parameters screen displayed in accordance with quality parameters tool procedures, in accordance with one or more aspects of the present disclosure.

FIG. 11A-11E illustrate a contract parameters screen displayed in accordance with contract parameters tool procedures, in accordance with one or more aspects of the present disclosure.

FIG. 12A-12C illustrate a review screen displayed in accordance with review tool procedures, in accordance with one or more aspects of the present disclosure.

FIG. 13A-13B illustrate a contract compare screen displayed in accordance with contract compare tool procedures, in accordance with one or more aspects of the present disclosure.

FIG. 14 a call flow for sending affinity data at a client layer, in accordance with one or more aspects of the present disclosure.

FIG. 15A-15B illustrate methods for VBC contract construction, in accordance with one or more aspects of the present disclosure.

FIG. 16 illustrates a system for VBC contract construction, in accordance with one or more aspects of the present disclosure.

FIG. 17 illustrates a computer system for VBC contract construction, in accordance with one or more aspects of the present disclosure.

FIG. 18 illustrates a cloud server for VBC contract construction, in accordance with one or more aspects of the present disclosure.

### DETAILED DESCRIPTION

Aspects of the present disclosure will now be described in detail with reference to the accompanying drawing figures. Like elements in the various figures may be denoted by like reference numerals. Further, in the following detailed description, specific details are set forth in order to provide a more thorough understanding of the claimed subject matter. However, it will be apparent to one of ordinary skill in the art that the aspects disclosed herein may be practiced without these specific details, or with details that are not described herein in the interest of clarity. Thus, in some instances, well-known features have not been described in detail to avoid unnecessarily complicating the description. Additionally, it will be apparent to one of ordinary skill in the art that the scale of the elements presented in the accompanying drawing figures may vary without departing from the scope of the present disclosure.

Aspects in accordance with the present disclosure generally relate to predictive analytic techniques for value based care (VBC), and more particularly to revenue forecasting using enhanced data processing techniques.

Systems and methods presented herein utilize an enhanced VBC system (e.g., enhanced contract builder system 100) that incorporates a sophisticated model builder. Specifically, systems and methods provided herein are capable of integrating various healthcare data, such as member (e.g., patient) data, claims, risk, premium, quality, and provider data, to facilitate the construction of comprehensive VBC models. The enhanced VBC system includes an affinity tool linked to an attribution tool, a model selection tool, a cost term tool, a quality parameter tool, a provider selection tool, a contract parameter tool, and a review tool. Leveraging an enhanced VBC system, aspects of the present disclosure may provide both descriptive and predictive analytics for efficient and accurate VBC contracting that is fit to account for future healthcare delivery and reimbursement scenarios. The ultimate goal of this VBC system is to construct one or more tailored payer-provider contracts that users can customize based on various inputs.

Implementation of systems and methods provided herein may allow users (e.g., healthcare administrators, actuaries, analysts, and providers) to quickly visualize VBC models and scenario plans, integrating data from various sources (e.g., EHRs, claims data, member-structured data files). This visualization may be the endpoint of an enhanced VBC system, allowing users to address critical operational issues in real-time, such as the impact of changing care delivery practices or adjusting reimbursement rates. This comprehensive approach ensures that the final output—tailored payer-provider contracts—can be customized in real-time to meet the specific needs and goals of the healthcare organization.

By enhancing the VBC model building process via an enhanced VBC system, extensive time (e.g., days, weeks, or months) that may be required by current systems to gather and analyze relevant data may be mitigated or eliminated. At the same time, the enhanced VBC system described herein may significantly increase the quality and fidelity of the models used, ensuring long-term reliability in various use cases. This approach not only improves efficiency but also enhances the ability of healthcare organizations to adapt to changing conditions and optimize their value-based care strategies. Ultimately, the system facilitates the creation of tailored provider contracts that can be customized based on real-time input, ensuring that the contracts are aligned with the specific needs and goals of the healthcare organization.

### Aspects Related to an Enhanced Value-Based Care Contract Builder System

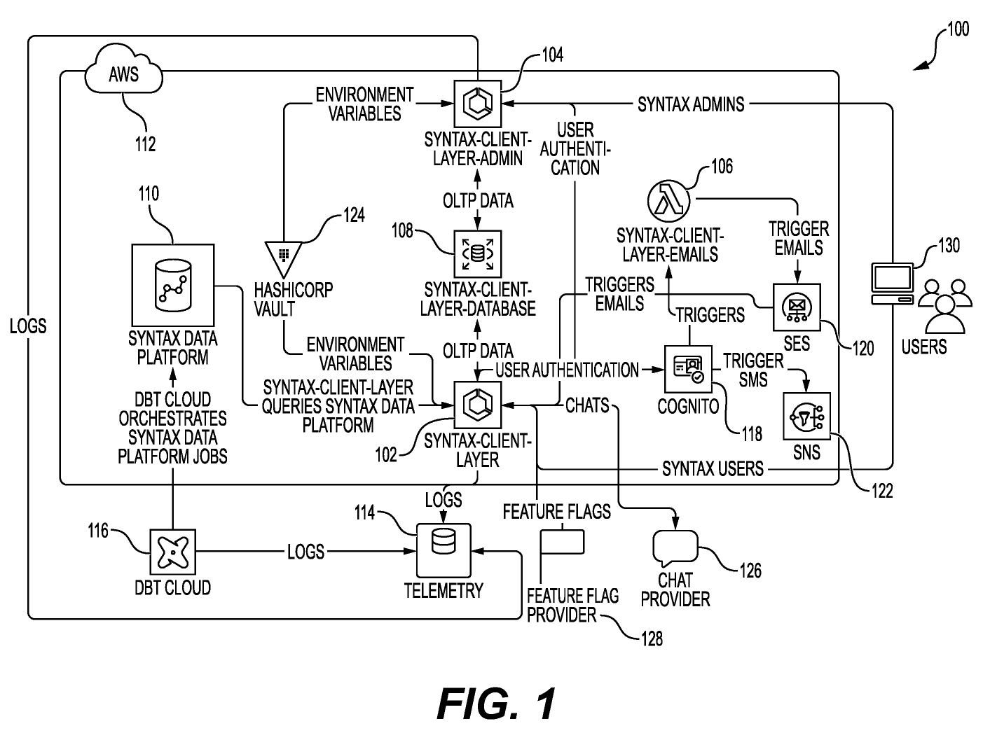

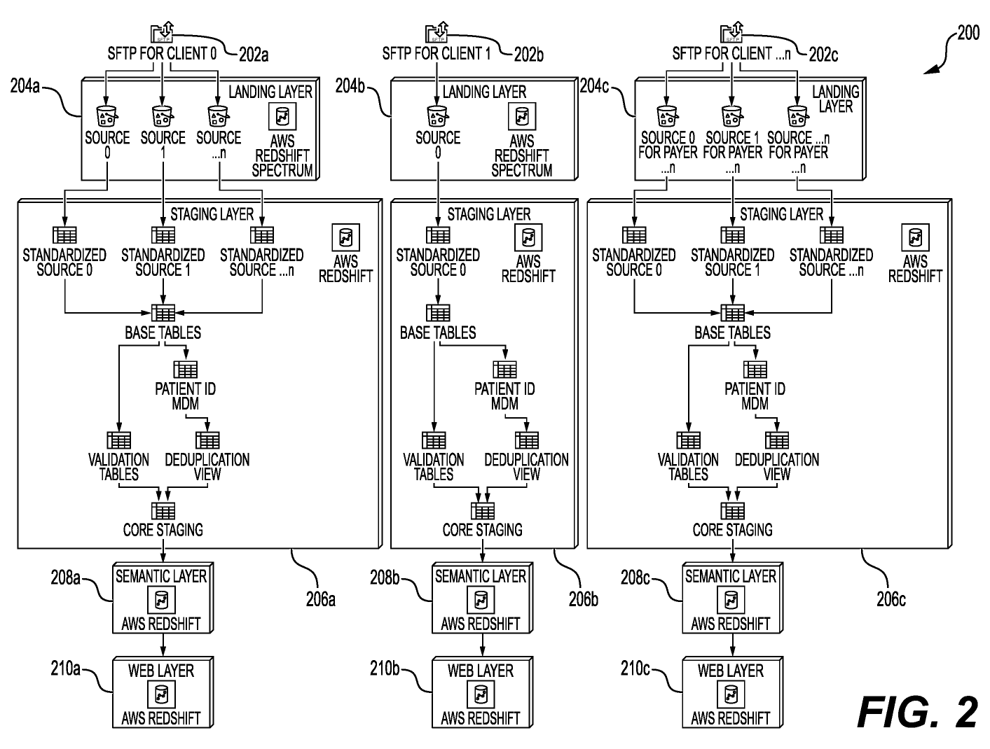

FIG. 1 illustrates an example of an enhanced VBC contract builder system (e.g., enhanced contract builder system 100), implemented according to at least one aspect of the present disclosure. In at least one aspect, the enhanced data processing architecture is configured or otherwise arranged to perform methods provided herein (e.g., method 1600). In at least one aspect, enhanced contract builder system 100 is configured or otherwise arranged to process data described in further detail with respect to FIG. 2. It may be understood that features of the data flow described in FIG. 2 are inherently and broadly described with respect to enhanced contract builder system 100.

Enhanced contract builder system 100 includes client layer 102, admin layer 104, email layer 106, database 108, primary data manager 110, cloud infrastructure layer 112, logging and monitoring layer 114, data pipeline layer 116, identity management layer 118, email service layer 120, notification service layer 122, secret management layer 124, chat manager 126, stream manager 128, and user interface 130. In at least one aspect, enhanced contract builder system 100 may be implemented on at least one memory and one or more processors having computer readable instructions stored thereon, which are capable of implementing enhanced VBC contract building schemes as part of enhanced contract builder system 100. In at least one aspect, client layer 102, admin layer 104, email layer 106, cloud infrastructure layer 112, logging and monitoring layer 114, data pipeline layer 116, identity management layer 118, email service layer 120, notification service layer 122, and/or secret management layer 124 may have at least one memory and one or more processors having computer readable instructions stored thereon, which are capable of implementing enhanced data processing schemes as part of the function of the enhanced data processing architecture. The one or more processor(s) of enhanced contract builder system 100 may be central processing units (CPUs). The one or more processor(s) of enhanced contract builder system 100 may be graphics processing units (GPUs). Where there is more than one processor, each processor may operate independently from one another or as part of the same network of controllers and/or systems. Where multiple processors are part of the same network of controllers and/or systems, they may operate in sequence with one another, in parallel with one another, as physical components of a shared virtual machine, or as components of server-less network capable of processing decomposed flow data.

In at least one aspect, a user may interact with client layer 102 to construct a VBC contract. Client layer 102 interacts with database 108 to store and retrieve VBC data. At or about the same time, client layer 102 interacts with primary data manager 110 (e.g., via query) to perform affinity, attribution, model type, cost, quality parameter, provider selection, and/or contract parameter operations. As a user interacts with client layer 102, admin layer 104 may manage (e.g., via one or more users) one or more environmental variables. Both client layer 102 and admin layer 104 may forward logs based on the environmental variable to logging and monitoring layer 114. Logging and monitoring layer 114 may compare the logs from admin layer 104 with logs obtained from client layer 102 and data pipeline layer 116 in order to accurately track jobs from client layer 102 processed at primary data manager 110. In at least one aspect, logs from data pipeline layer 116 reflect orchestration operations used to queue jobs at primary data manager 110.

In at least one aspect, user access to client layer 102 may be moderated using identity management layer 118. Identity management layer 118 may also invoke activity at an email layer 106, which is configured or otherwise arranged to construct emails based on operations between client layer 102 and/or primary data manager 110 and send the emails via email service layer 120.

In at least one aspect, chat manager 126 may facilitate user communication with another party via a network (not shown).

In at least one aspect, cloud infrastructure layer 112 may host any one of the following, alone or in combination:
client layer 102, admin layer 104, email layer 106, database 108, primary data manager 110, identity management layer 118, email service layer 120, notification service layer 122, secret management layer 124. Cloud infrastructure layer 112 may be at a container management unit (not shown). Cloud infrastructure layer 112 provides the underlying infrastructure for all cloud-based components, ensuring their security and functionality.

The architecture includes client layer 102 (e.g., syntax-client-layer), located on a container management unit (e.g., Amazon Web Services (AWS) Elastic Container Service (ECS), Kubernetes, Docker, Helm, Prometheus), which may provide a user with enhanced value-based care (VBC) contract procedures and managing contract negotiations. Client layer 102 may be accessed via a web browser. It may be implemented using a front-end or full development stack (e.g., React tech, Angular). Individual users may be authenticated at client layer 102 using a username, password, and multi-factor authentication (MFA) with a one-time password (OTP) managed by an identity manager (e.g., AWS Cognito, Node.js, Auth0). In some cases, syntax-client-layer 102 may be used to create and add users to arbitrary groups, each with their own custom permissions. This may allow a user to set up approval chains and workflows.

The architecture includes admin layer 104 (e.g., syntax-client-layer-admin), located on the container management unit, which may provide admin console functionality for enhanced VBC contract procedures. It may be implemented using a front-end development stack. Individual users may be authenticated at admin layer 104 with a username, password, and MFA with OTP managed by the identity manager. Authorization is based on user roles.

The architecture includes email layer 106 (e.g., syntax-client-layer-emails), located on a serverless computing manager (e.g., AWS Lambda, Google Cloud Functions), which may be used to send emails for user account creation and password resets. It may be implemented using a back-end development stack (e.g., Node.js). Service accounts may authenticate through an identity and access management (IAM) policy that allows the identity manager to trigger email layer 106.

The architecture includes database 108 (e.g., syntax-client-layer-database), located on a relational database unit (e.g., AWS RDS, Google Cloud SQL), which may store all transactional data for enhanced VBC contract procedures. It may be implemented using a database management system (e.g., PostgreSQL, MySQL). Individual users may authenticate with a username and password, and the database management system handles authorization. Service accounts may also authenticate using a username and password, with the database management system handling authorization. In at least one aspect, database 108 may include one or more of relational databases (RDBMS) which use structured query language (SQL) for data management, NoSQL databases (e.g., document stores, key-value stores, column-family stores, graph databases), in-memory databases, NewSQL databases, time-series databases, object-oriented databases, hierarchical databases, network databases, distributed databases, cloud databases, multimodel databases, embedded databases, and the like. Database 108 may be treated as a data warehouse, data lake, data well, or another storage system. Database 108 may be optimized for querying and analysis, allowing the enhanced methods described herein (e.g., method 1600).

The architecture includes primary data manager 110 (e.g., Syntax Data Platform), located on a data warehousing unit (e.g., AWS Redshift, Google BigQuery), which may store healthcare claims and other client-provided data, providing analytical capabilities for enhanced VBC contract procedures. It may be implemented using a data warehousing stack. Individual users may authenticate with a username and password, and the data warehousing unit handles authorization. Service accounts may also authenticate using a username and password, with the data warehousing unit handling authorization. In at least one aspect, primary data manager 110 may be split into its own threat model.

The architecture includes cloud infrastructure layer 112 (e.g., Syntax Cloud Infrastructure), located on a cloud infrastructure unit (e.g., AWS, Google Cloud Platform), which may provide base-level cloud infrastructure such as networking, load balancers, web application firewalls (WAF), and threat detection services. Individual users may authenticate with a username, password, and MFA. IAM may handle authorization for user requests. Service accounts may authenticate using an access key and secret key, with IAM handling authorization. In at least one aspect, cloud infrastructure layer 112 may be split into its own threat model.

The architecture includes logging and monitoring layer 114 (e.g., Datadog), which may provide logs, application performance monitoring, and the like. Individual users may authenticate with a username and password managed by logging and monitoring layer 114. Service accounts may authenticate using an application programming interface (API) key and token managed by logging and monitoring layer 114. In some cases, logging and monitoring layer 114 may be implemented as SaaS tools communicatively coupled to but independent from infrastructure layer 112.

The architecture includes data pipeline layer 116 (e.g., DBT Cloud), which may execute data pipelines in the data platform. Individual users may authenticate with a username and password, with permissions managed within data pipeline layer 116. In some cases, pipeline layer 116 may be implemented as SaaS tools communicatively coupled to but independent from infrastructure layer 112.

The architecture includes identity management layer 118 (e.g., AWS Cognito, Auth0), which may be used for user identity and access management. Individual users may authenticate with a username, password, and MFA. Identity and access management (IAM) handles authorization for user requests. Service accounts may authenticate using an access key and secret key, with IAM handling authorization. In at least one aspect, access to identity management layer 118 is the same as access to cloud infrastructure layer 112.

The architecture includes email service layer 120 (e.g., Simple Email Service (SES)), which may facilitate sending emails to users as part of registration and password resets. Individual users may authenticate with a username, password, and MFA. Identity and access management (IAM) handles authorization for user requests. Service accounts may authenticate using an access key and secret key, with IAM handling authorization. In at least one aspect, access to email service layer 120 is the same as access to cloud infrastructure layer 112. In some cases, email service layer 120 may send notifications regarding different activity in an application implemented at the architecture.

The architecture includes notification service layer 122 (e.g., Simple Notification Service (SNS)), which may facilitate sending SMS messages to clients choosing SMS as their MFA method. Individual users may authenticate with a username, password, and MFA. Identity and access management (IAM) handles authorization for user requests. Service accounts may authenticate using an access key and secret key, with IAM handling authorization. In at least one aspect, access to notification service layer 122 is the same as access to cloud infrastructure layer 112.

The architecture includes secret management layer 124 (e.g., Syntax Vault, AWS Secrets Manager), which may store environment variables used by various components, including credentials to database layer 108 and primary data manager 110. Individual users may authenticate with GitHub authentication. Service accounts may authenticate using cloud infrastructure service credentials. At deployment time, the application accesses secret management layer 124 to retrieve updated environment variables. Identity and access management (IAM) handles authorization for service account requests. In at least one example, secret management layer 124 may perform operations at deploy time. When code is deployed to client layer 102, an environment file may be generated from reading information at secret management layer 124 as part of deployment. The environment file may then be used to populate environment variables at client layer 102 and admin layer 104.

The architecture includes chat manager 126 (e.g., Stream), which may provide chat channel functionality. Individual users may authenticate with a username, password, and MFA with OTP managed by identity management layer 118. Client layer 102 is responsible for authorizing user activity based on the user’s role and organization membership. Service accounts may authenticate using an API key and token managed by chat manager 126. This component is in scope for the threat model. In some cases, chat manager 126 may be implemented as SaaS tools communicatively coupled to but independent from infrastructure layer 112.

The architecture includes a feature flag manager 128. Feature flag manager 126 may be LaunchDarkly or another suitable tool for feature flag management. Client-layer 102 may make requests to feature flag manager 126 for feature flags, which the client layer 102 may use for configuration and conditional rendering of different functionality.

In at least one aspect, user interface 130 may be implemented at a device communicatively coupled to cloud infrastructure layer 112. Said device may have a display. A user can enter control inputs through user interface 130 (such as a keyboard, microphone, or touchscreen). In one example, a device having user interface 130 can include, but is not limited to, a mobile computing device (such as a smartphone or tablet computer), wearable computing device (such as a smart watch or headset), a desktop computer, laptop computer, set-top box, smart television, smart display screen, kiosk, or other type of computing device having at least one processor and computer-readable memory. In addition to at least one processor and memory, such a computing device may include software, firmware, hardware, or a combination thereof. Software may include one or more applications, a browser, and an operating system. Hardware can include, but is not limited to, a processor, memory, display or other input/output device. A communication interface and transceiver can be included to perform data communication (wired or wireless) over network, such as the network described below. In at least one aspect, user interface 130 may be implemented at a remote computing device configured or otherwise arranged to implement systems and methods provided herein.

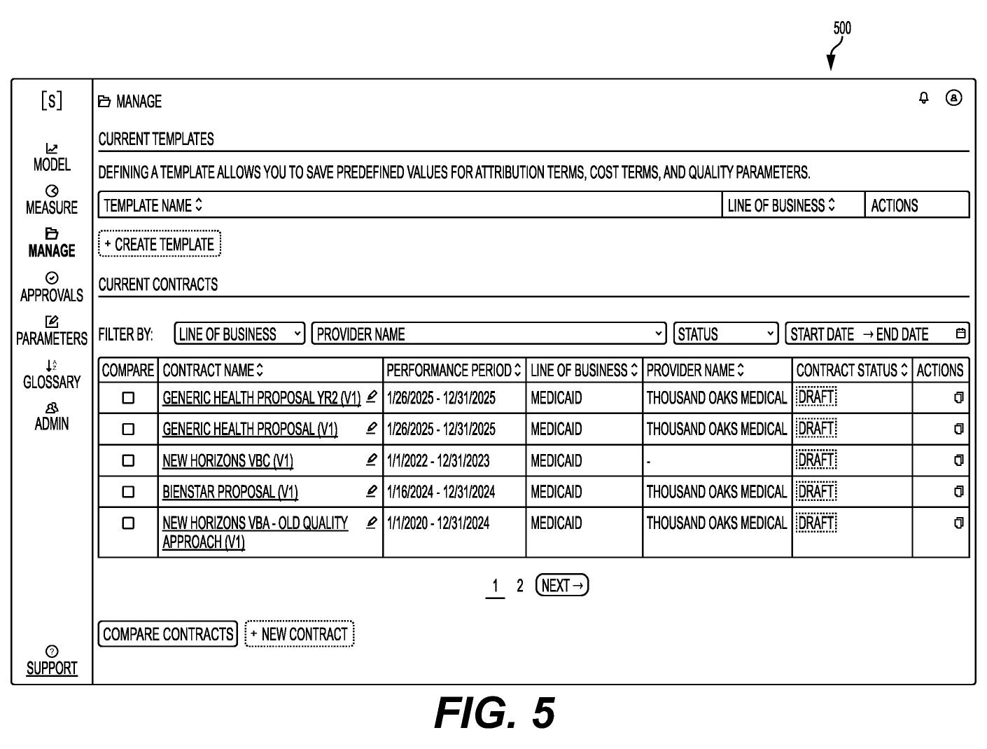

In some embodiments, a user may access enhanced contract builder system 100 via a browser accessible via user interface 130. A user may be able to access a contract builder tool via user interface 130, where the contract builder tool may be an application, such as, an application running under control of an operating system on a computational device or a web application operating which may be accessed via a network and via a browser. Contract builder tool may be configured or otherwise arranged to access data from enhanced contract builder system 100 to, for example, display VBC contract informatics. In at least one aspect, VBC contract informatics may represent a final output of models constructed at enhanced contract builder system 100. Visualization may be achieved by a visualizer module. The visualizer module may allow a computational device to generate interactive dashboards at a user interface 130 where users can explore the data and pull levers to see the impact of various actuarial inputs and model selections. Examples of VBC contract informatics are further discussed with respect to FIGS. 5-12.

In at least one aspect, enhanced contract builder system 100 may be implemented on a network. In at least one aspect, the network may be practically implemented as one or more networks. The network may be a wired network, a wireless network, or a combination of both. In at least one aspect, a wireless network may include a wireless local area network (WLANs) (e.g., a wireless fidelity (Wi-Fi) network), a wireless personal area networks (WPANs) (e.g., a Bluetooth network, a Zigbee network), a wireless metropolitan area network (WMANs) (e.g., a worldwide interoperability for microwave access (WiMAX) network), a wireless wide area network (WWANs) (e.g., a fourth generation long-term evolution (4G LTE) network, a fifth generation new radio (5G NR) network, satellite networks, mesh networks, ad hoc networks, near field communication (NFC) networks, infrared (IR) communication networks, ultra-wideband (UWB) networks, long range wide area networks (LoRaWAN) (e.g., a network optimized for Internet-of-Things (IoT) applicability), and the like.

In at least one additional aspect, the enhanced data processing architecture of FIG. 1 may utilize serverless computing, allowing the architecture to implement custom features (e.g., custom code) in response to specific events, such as data updates or user actions.

### Example Data Flow through an Enhanced Value-Based Care Contract Builder System

FIG. 2 illustrates an example data flow 200 through an enhanced contract builder system (e.g., enhanced contract builder system 100). Data flow 200 is illustrated by way of data for Client 0, Client 1, and Client n.

Data flow 200 for Client 0 begins at a secure file transfer protocol (SFTP) 202a and proceeds to a landing layer 204a, where data is transferred to Sources 0, 1, . . ., n. Data is then transferred to a staging layer 206a. At the staging layer 206a, data from each of Sources 0, 1, . . ., n is delivered to Standardized Sources 0, 1, . . . n, respectively. Data from each Standardized Source is combined at a base table. The base table is then divided into a validation table and a duplication view, where the duplication view is combined with a member identification directory (member ID MDM). The duplication view and the validation table are then recombined to construct a core staging table. The staging layer 206a delivers the core staging table to a semantic layer 208a, which then delivers the core staging table to the web layer 210a.

Data flow 200 for Client 1 begins at a SFTP 202b and proceeds to a landing layer 204b, where data is transferred to Sources 0. Data is then transferred to a staging layer 206b. At the staging layer 206b, data from each of Source 0is delivered to Standardized Source 0. Data from Standardized Source 0 is converted to a base table. The base table is then divided into a validation table and a duplication view, where the duplication view is combined with a member identification directory (member ID MDM). The duplication view and the validation table are then utilized to construct a core staging table. The staging layer 206b delivers the core staging table to a semantic layer 208b, which then delivers the core staging table to the web layer 210b.

Data flow 200 for Client 0 begins at a secure file transfer protocol (SFTP) 202c and proceeds to a landing layer 204c, where data is transferred to Sources 0, 1, . . ., n. Data is then transferred to a staging layer 206c. At the staging layer 206c, data from each of Sources 0, 1, . . . .,is delivered to Standardized Sources 0, 1, . . . n, respectively. Data from each Standardized Source is combined at a base table. The base table is then divided into a validation table and a duplication view, where the duplication view is combined with a member identification directory (member ID MDM). The duplication view and the validation table are then utilized to construct a core staging table. The staging layer 206c delivers the core staging table to a semantic layer 208c, which then delivers the core staging table to the web layer 210c.

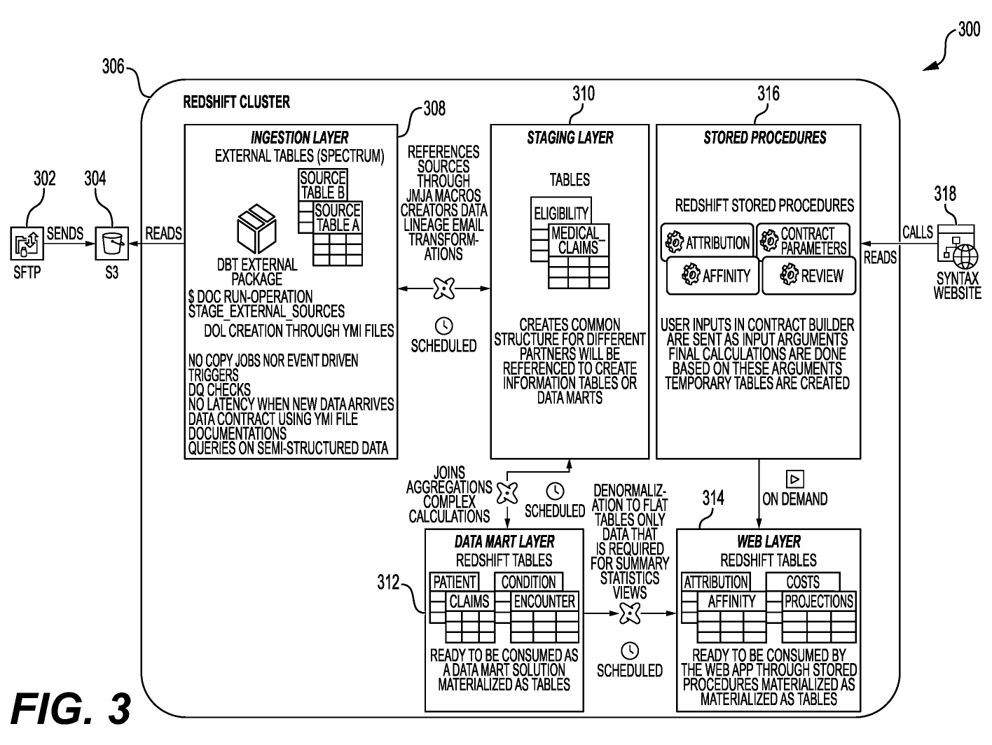

FIG. 3 illustrates an example data flow 300 through an enhanced contract builder system (e.g., enhanced contract builder system 100). Data flow 300 may be considered to have substantial overlap with data flow 200. Data flow 300 begins with SFTP 302 transferring data to a readable data sink 304. A cluster 306 implemented as part of a computer system (e.g., a Redshift cluster) may read the data at cluster 306 by way of an ingestion layer 308. Ingestion layer 308 reads the data as source tables and relays the source tables to a staging layer 310. The staging layer 310 may process the source tables to generate eligibility and medical claim tables, which may be considered intermediate tables or “data mart" sources within data flow 300. A data mart layer 312 retrieves the intermediate tables and processes the tables to construct member, claims, condition, and encounter tables. Data mart layer 312 then forwards the member, claims, condition, and encounter tables to a web layer 314. Web layer 314 may be accessed on demand by a stored procedures module 316. The stored procedures module 316 may store procedures associated with, for example, attribution, affinity, contract parameters, and review. Each of the stored procedures may be implemented using data accessed on web layer 314. A website 318 (e.g., accessed via user interface 130) may call the stored procedures module 316 to execute any of the procedures stored therein.

### Aspects Related to a Toolkit for VBC Contract Construction

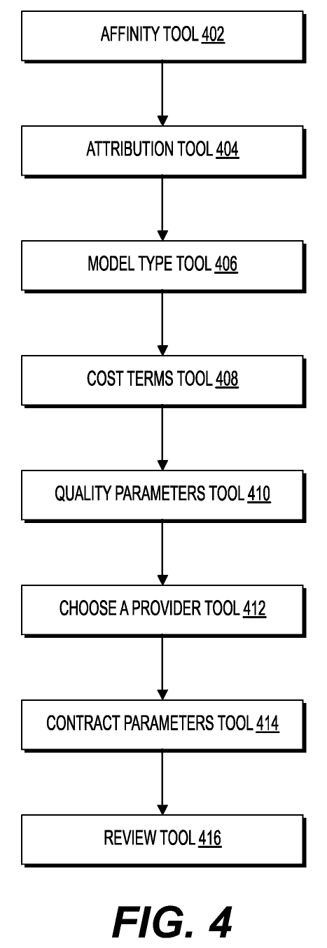

FIG. 4 illustrates an example enhanced VBC contract construction toolkit 400, which may be implemented according to at least one aspect of the present disclosure. In at least one aspect, enhanced VBC contract construction toolkit 400 may be configured or otherwise arranged to be implemented on an enhanced contract builder system (e.g., enhanced contract builder system 100). In at least one aspect, enhanced VBC contract construction toolkit 400 may be configured or otherwise arranged to process data described in further detail with respect to FIG. 2. It may be understood that features of the data flow described in FIG. 2 are inherently and broadly described with respect to enhanced VBC contract construction toolkit 400.

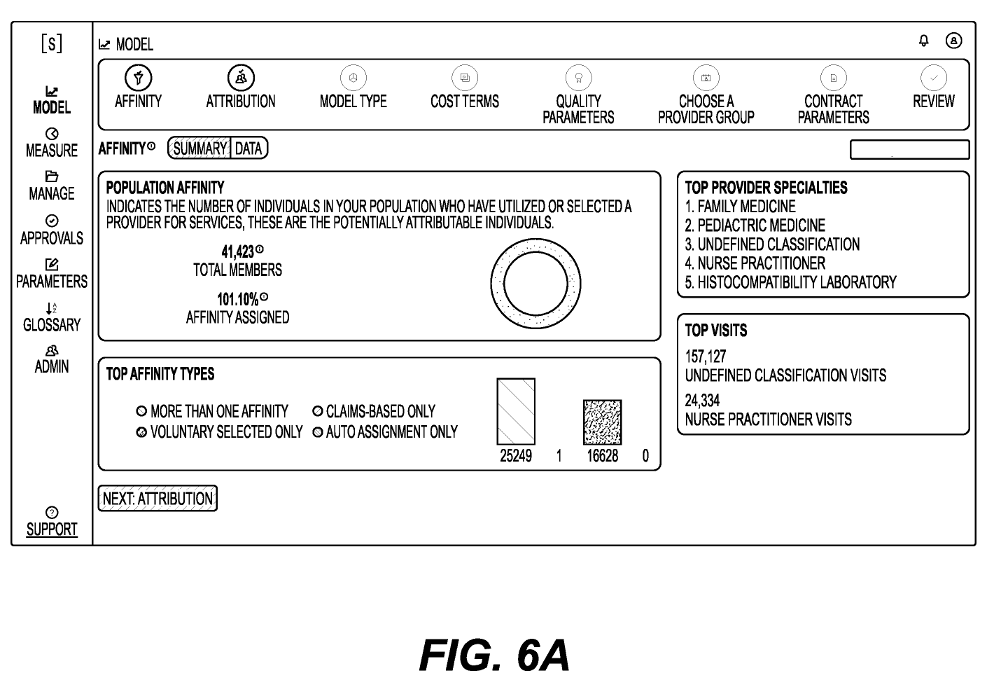

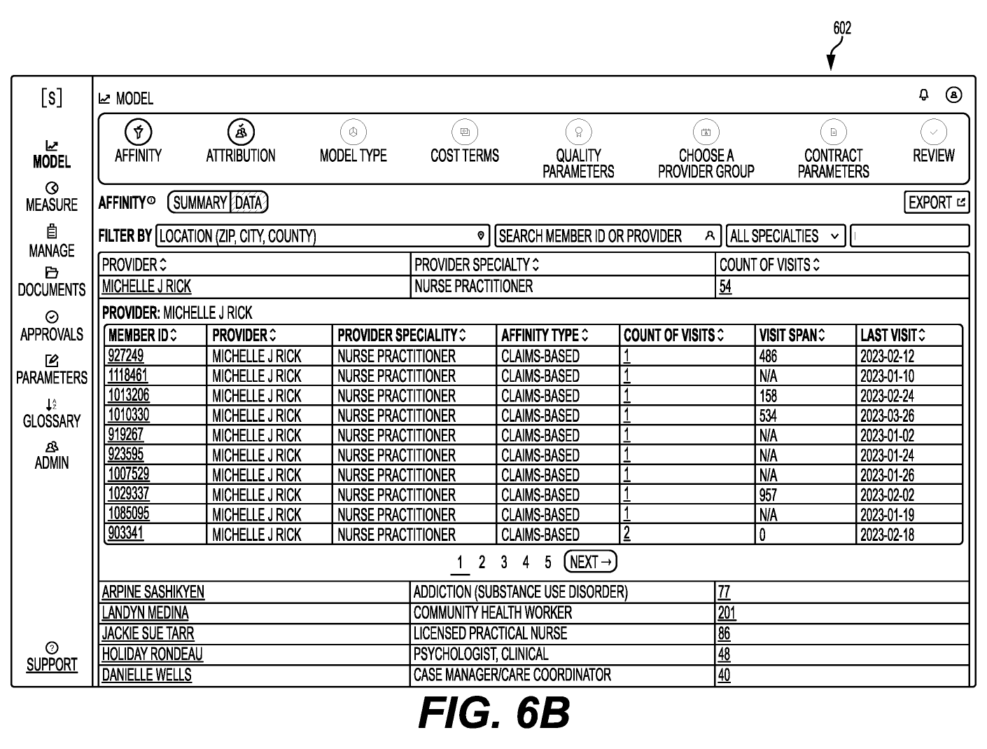

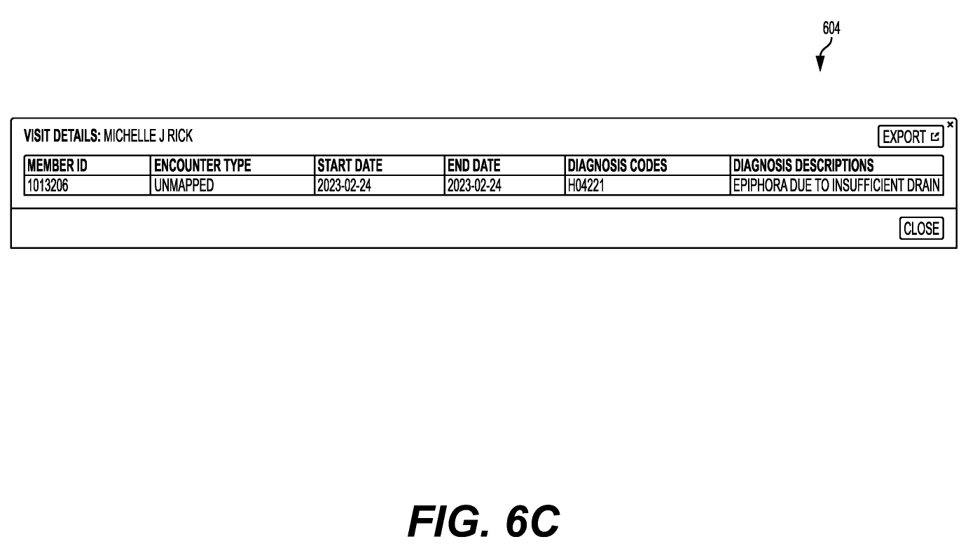

Enhanced VBC contract construction toolkit 400 may streamline the process of selecting and managing contracts through a series of specialized tools, each addressing an aspect of the contracting workflow. To perform methods provided herein, enhanced VBC contract construction toolkit 400 may first implement an affinity tool 402. Affinity tool 402 may utilize several key parameters to accurately relate members to providers. In one example, a member ID may serve as a unique identifier for each member. In one example, a provider parameter may specify the healthcare professional responsible for managing the member's care. This may include details about the provider's specialty, which may determine the type of care the member receives. The provider’s specialty may help tailor care to the member's specific health needs, enhancing the accuracy of the attribution process. In one example, an “affinity type” parameter may define the nature of the relationship between the member and the provider. This may include voluntary selection by the member, assignment by a payer, or referral from another healthcare professional. The count of visits parameter may measure the frequency of interactions between the member and the provider. This count may indicate the strength of the relationship, with a higher number of visits suggesting a more established connection. The visit span parameter may refer to the duration over which the member has been seeing the provider, reflecting the continuity of care provided. A “last visit” parameter may capture the most recent interaction between the member and the provider. This information may provide insight into the current status of the member's health and the immediacy of the care relationship. The timing of the last visit may impact the provider's ability to manage the member's care effectively. By implementing these parameters—member ID, provider, provider specialty, affinity type, count of visits, visit span, and last visit—an affinity tool may accurately relate members to providers. Affinity tool 402 may be further understood with respect to the example user screen of FIGS. 6A-6C.

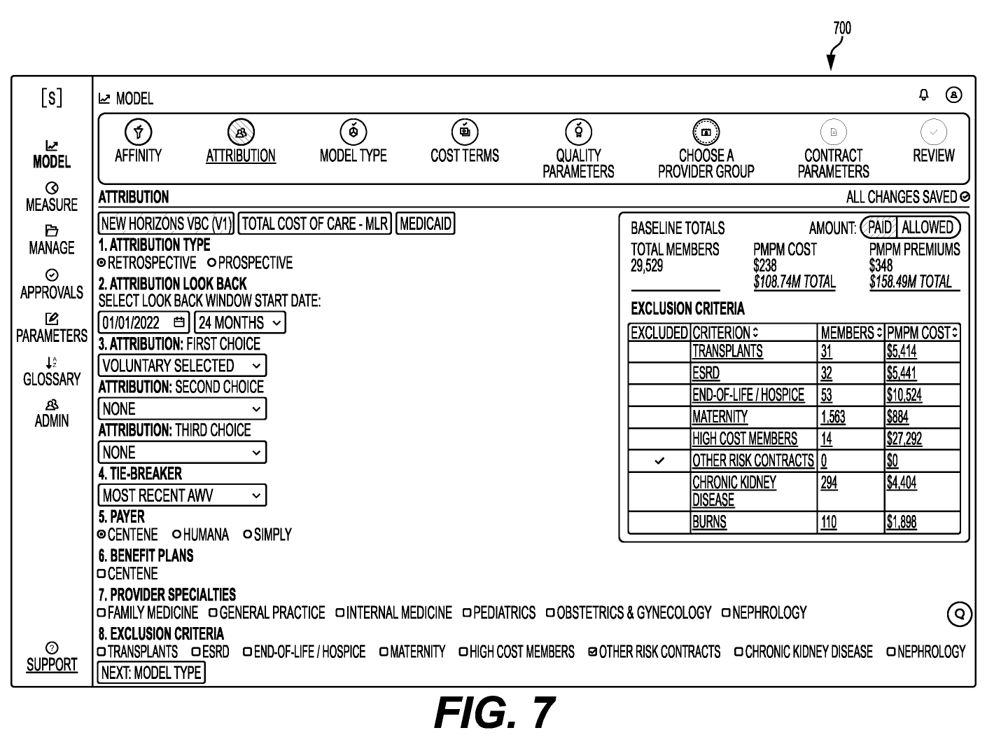

Enhanced VBC contract construction toolkit 400 may deliver information to an attribution tool 404. Attribution tool 404 may utilize several parameters to assign members to providers. In one example, an attribution type parameter may define whether the attribution is retrospective, based on care provided during the contract year, or prospective, based on data from the prior calendar year. An attribution lookback parameter may specify the time frame, ranging from 1 to 5 years, used for analyzing data when performing attribution. An attribution first choice parameter may identify the primary method for attributing members to providers, such as claims-based, voluntary assignment, or auto assignment. If the first choice is not applicable, the attribution second choice and attribution third choice parameters may provide secondary and tertiary methods for member attribution. A tie breaker parameter may be used when there is a tie between attribution choices, employing methods such as Most Recent Plurality, Most Recent Annual Wellness Visit (AWV), or custom user-specified methods. A payer (e.g., insurer) parameter may identify the insurance provider responsible for the member's coverage, while a benefit plan parameter may specify the member's insurance plan, influencing the attribution criteria and the scope of covered services. A provider specialty parameter may facilitate member attribution to providers with the appropriate expertise, such as Family Practice, Internal Medicine, or Pediatrics. An exclusion criteria parameter may identify members who should be excluded from attribution due to high costs, unusual circumstances, or types of care not covered by the performance model. By implementing these parameters—attribution type, attribution lookback, attribution first choice, attribution second choice, attribution third choice, tie breaker, payer, benefit plan, provider specialty, and exclusion criteria—an attribution tool may accurately assign members to providers. In at least one example, a user may access attribution tool 404 in accordance with procedures provided herein to make selection on the left side of the screen. As a result of the selections, summary statistics and information may be calculated in real-time and displayed on the right side of the screen. Attribution tool 404 may be further understood with respect to the example user screen of FIG. 7.

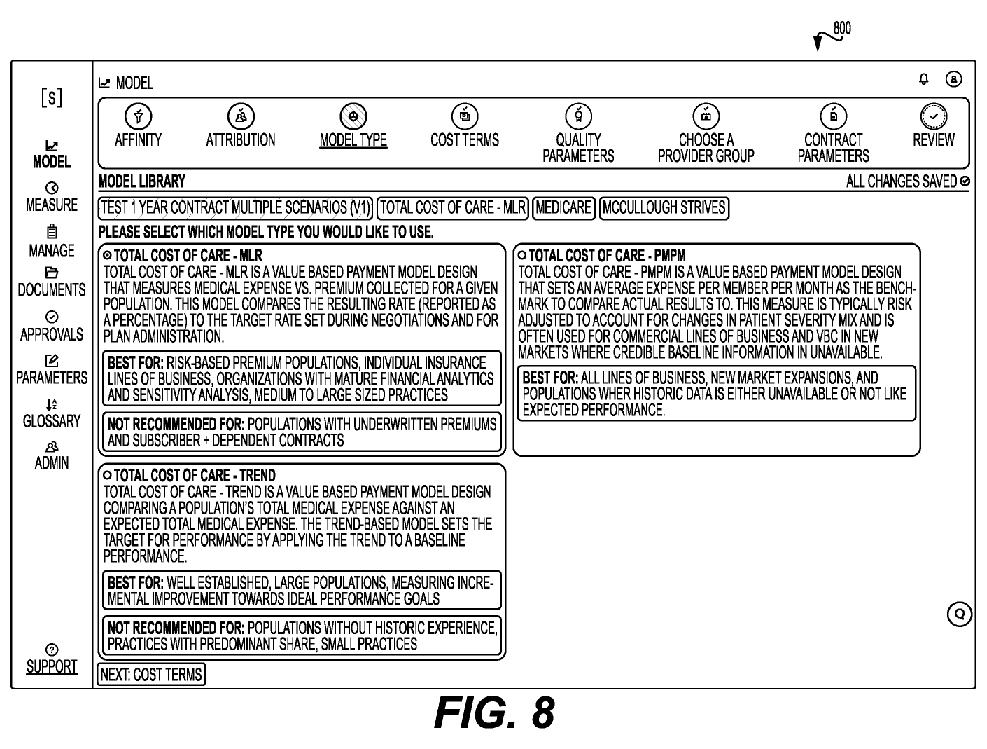

Enhanced VBC contract construction toolkit 400 may deliver information from attribution tool 404 to a model type tool 406. In one example, a model type parameter may specify the financial model to be used, such as medical loss ratio (MLR), per member per month (PMPM), or trend-based models. An MLR model may focus on the ratio of medical expenses to total revenue, ensuring that a certain percentage of revenue is spent on member care. In one example, a PMPM model sets a target for medical and/or pharmacy expense alone, without consideration for premiums. A trend model may analyze historical cost data to project future expenses and set benchmarks for performance. By implementing these parameters—model type, MLR, PMPM, and trend—model type tool 406 may determine the most suitable financial model for VBC contracts. Model type tool 406 may be further understood with respect to the example user screen of FIG. 8.

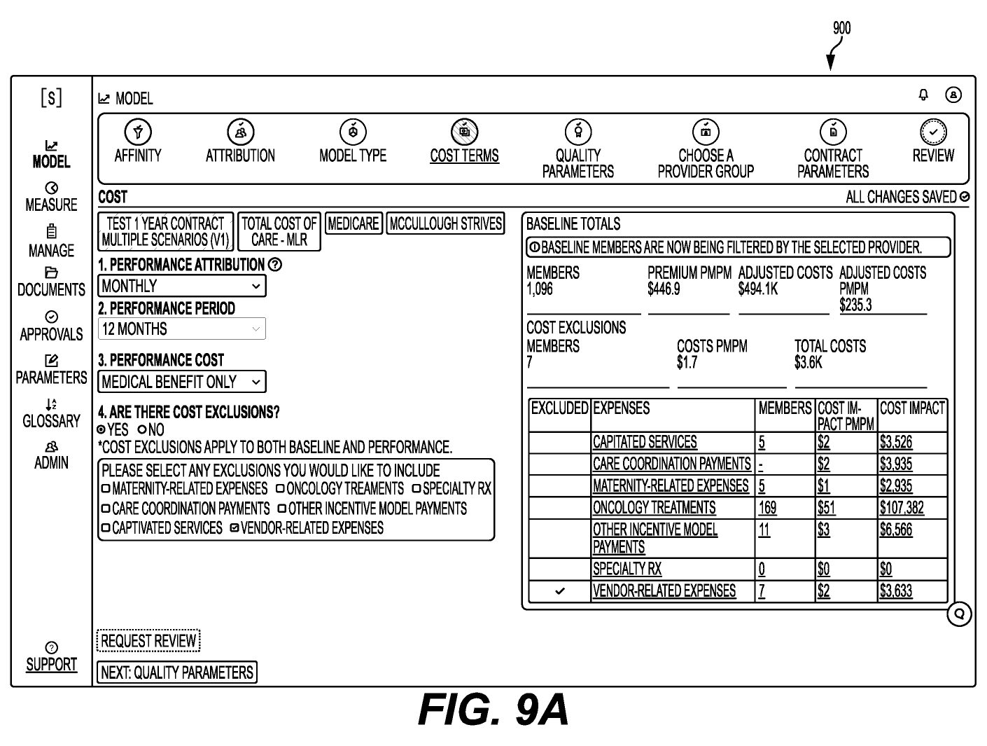

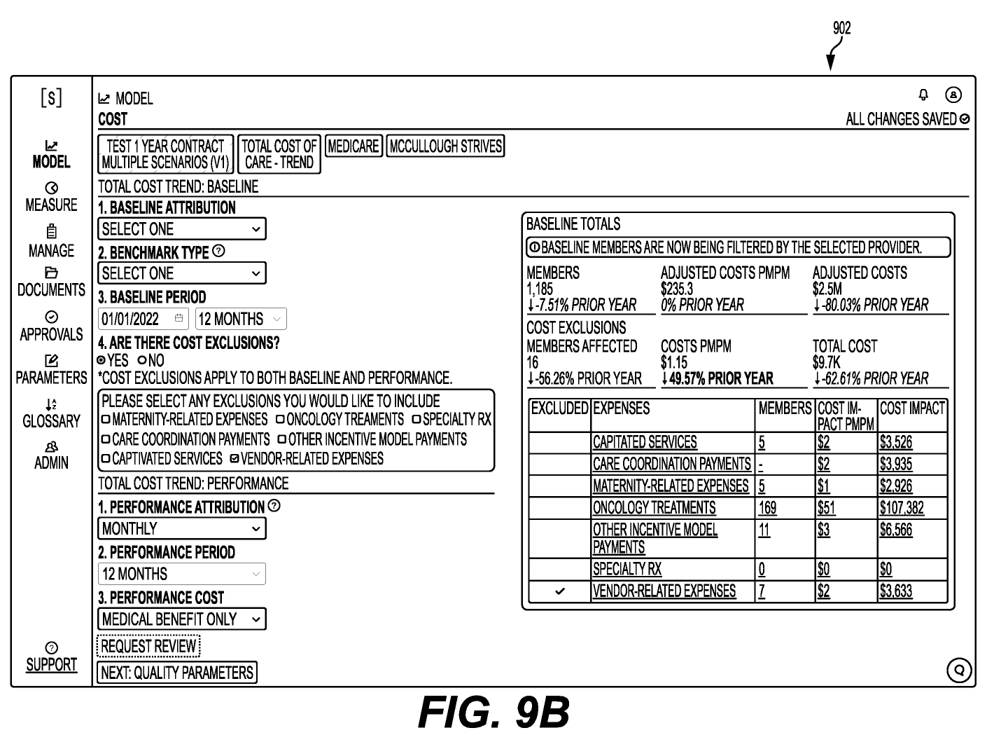

Enhanced VBC contract construction toolkit 400 may deliver information from model type tool 406 to cost terms tool 408. Cost terms tool 408 may utilize several parameters to determine and manage the cost aspects of VBC contracts. In one example, a baseline attribution parameter may define the initial assignment of members to providers, establishing the starting point for cost calculations. A benchmark type parameter may specify the standard or reference point against which performance will be measured, such as historical costs or industry standards. A baseline period parameter may identify the time frame used to establish the baseline costs, ranging from one to several years. A cost exclusions parameter may identify specific costs that should be excluded from the calculations, such as high-cost cases, unusual circumstances, or types of care not included in the performance model. In one case, a performance attribution parameter may define how members are assigned to providers during the performance period, ensuring that the same criteria are applied consistently. In one case, a performance attribution parameter may attributed monthly. In one case, a performance attribution parameter may be attributed annually. A performance period parameter may specify the duration over which the provider's performance is evaluated, which can influence the assessment of cost management and care quality. A performance cost parameter may calculate the actual costs incurred during the performance period, comparing them to the baseline and benchmark to determine the provider's financial performance. By implementing these parameters—baseline attribution, benchmark type, baseline period, cost exclusions, performance attribution, performance period, and performance cost—cost terms tool 408 may determine and manage the financial aspects of VBC contracts. Cost terms tool 408 may be further understood with respect to the example user screen of FIGS. 9A-9B.

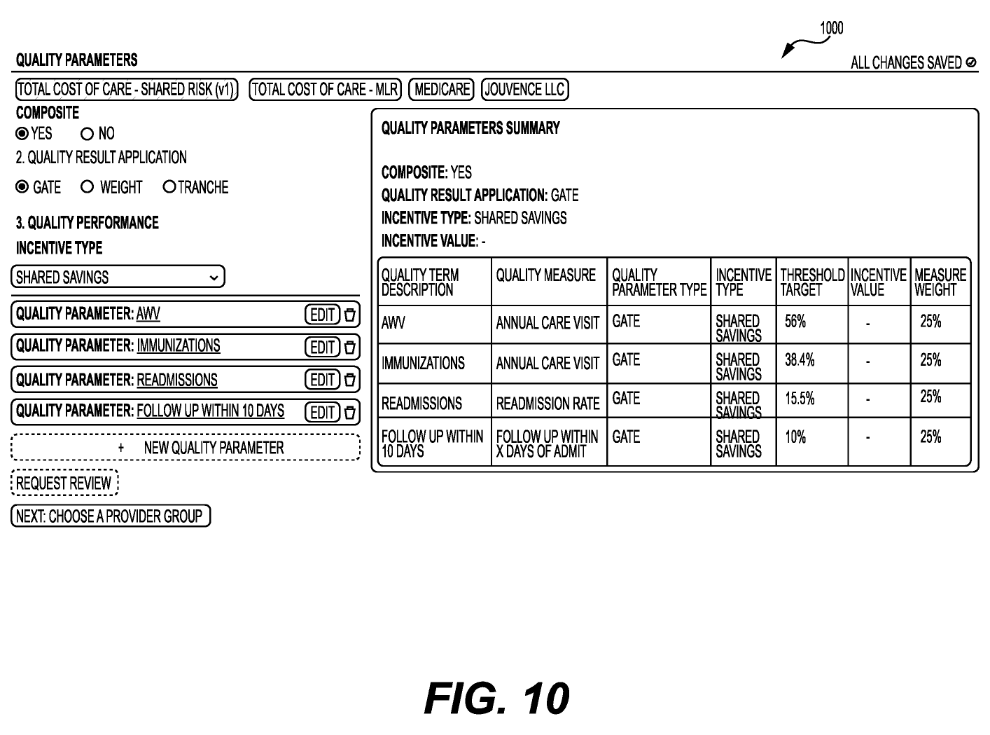

Enhanced VBC contract construction toolkit 400 may deliver information from cost terms tool 408 to a quality parameter tool 410. Quality parameter tool 410 may utilize several parameters to evaluate and manage the quality of care provided. In one example, a composite parameter may define whether multiple quality measures are combined into a single score. A quality result application parameter may specify how the quality results are applied, such as gate (threshold that must be met), weight (relative importance of each measure), or tranche (tiered levels of performance). A quality performance incentive type parameter may identify the type of incentive provided for meeting quality measures. Example quality parameters may include AWV, immunizations, readmissions, follow-up within 10 days, and similar measures. By implementing these parameters—composite, quality result application, and quality performance incentive type—Quality Parameters tool 410 may accurately evaluate and manage the quality of care provided. Quality Parameters tool 410 may be further understood with respect to the example user screen of FIG. 10.

Enhanced VBC contract construction toolkit 400 may deliver information from quality parameter tool 410 to a choose a provider tool 412. Choosing a provider tool 412 may allow a user to select one or more providers to be considered within the contract builder tool.

Enhanced VBC contract construction toolkit 400 may deliver information from choose a provider tool 412 to a contract parameters tool 414. Contract parameters tool 414 may utilize several parameters to define the terms of VBC contracts based on the selected financial model. For an MLR model, the parameters may include itemized adjusters such as administrative expense add-ons, claim corrections, and incurred but not reported (IBNR) claims. Performance stop loss and its associated negotiation range may be specified to limit financial risk. Performance pooling may be indicated with a yes or no parameter. The MLR target, floor, and ceiling may be defined along with their negotiation ranges. A premium adjuster parameter may be included to account for variations in premium calculations, or various insufficiencies in underlying data related to premium calculations. The shared savings/loss rate and its negotiation ranges may be specified, along with a parameter to indicate whether shared savings/losses are applied. Maximum shared savings and maximum shared loss parameters may be set to cap the financial exposure. Coordination monthly payments may be indicated with a yes or no parameter. A care coordination payment base rate and its negotiation range may be defined, with an option to indicate whether the payment is risk-adjusted. The maximum member-level care coordination payment amount may be specified, along with the maximum care coordination fee per member.

Contract parameters are user-customizable for any given contract. In a similar manner, actuarial parameters may be user-customizable. Specifically, a user can set organizational-level default values for actuarial parameters, and may override those defaults and customize the values for certain contracts.

Different contracts might need different actuarial parameters. For example, Thousand Oaks Medical might want to use one expense trend when modeling contracts with Canticle, while another set of expense trend values might be desired when modeling contracts with Point Health. As another example, BHS might want to model a contract for a prior year, such as 2023, to evaluate the accuracy of calculations. BHS might also want to model a contract for an upcoming year, such as 2025. This kind of functionality is possible if actuarial parameters can be uniquely defined as part of each contract. This is because the differing performance years in the prior examples would require different blending weights, these blending weights may be defined at a per-contract level. Additionally, actuaries will want to experiment with different potential actuarial parameters or change actuarial parameters across the year. For example, Incurred But Not Reported (IBNR) for a given year increases until it finally reaches 1.

Systems and methods provided herein provide dynamic actuarial parameters for forward propagation through a model. It is more efficient to make actuarial parameters a self-serve function that a user may configure. This has the benefit of decoupling data integration from configuration and removing system dependency on analytics to complete technical testing. By continuously updating and adjusting actuarial assumptions based on real-time data and emerging trends, such a system enhances the accuracy and relevance of predictive models. This adaptability allows organizations to respond swiftly to changing conditions, such as economic fluctuations, demographic shifts, or new regulatory requirements. The dynamic nature of these parameters may help identify potential risks and opportunities earlier, thereby improving overall financial stability and resilience.

For a PMPM model, the contract parameters may include complete months and itemized adjusters such as administrative adjustments, IBNR, and claim corrections. Performance stop loss and its negotiation range may be specified, with an option to suggest a range. Performance pooling may be indicated with a yes or no parameter. The PMPM target may be set, with an option to specify targets at the benefit level. In one example, exclusive provider organization (EPO), health maintenance organization (HMO), point of service (POS), and preferred provider organization (PPO) PMPM targets may be defined along with their negotiation ranges. A random variability threshold risk adjuster may be included to account for fluctuations in member costs. The shared savings/loss rate and its negotiation ranges may be specified, along with a parameter to indicate whether limits are applied to shared cost savings. Maximum shared savings PMPM and maximum shared loss PMPM parameters may be set with their negotiation ranges. Care coordination monthly payments may be indicated with a yes or no parameter. A base rate (e.g., a care coordination base rate) and its negotiation range may be defined, with an option to indicate whether the payment is risk-adjusted. The maximum member-level care coordination payment amount may be specified, along with the maximum care coordination fee per member. A maximum amount and its negotiation range may be set, with a suggested maximum amount PMPM parameter included. The negotiation ranges for these parameters may be predetermined or determined during contract negotiation.

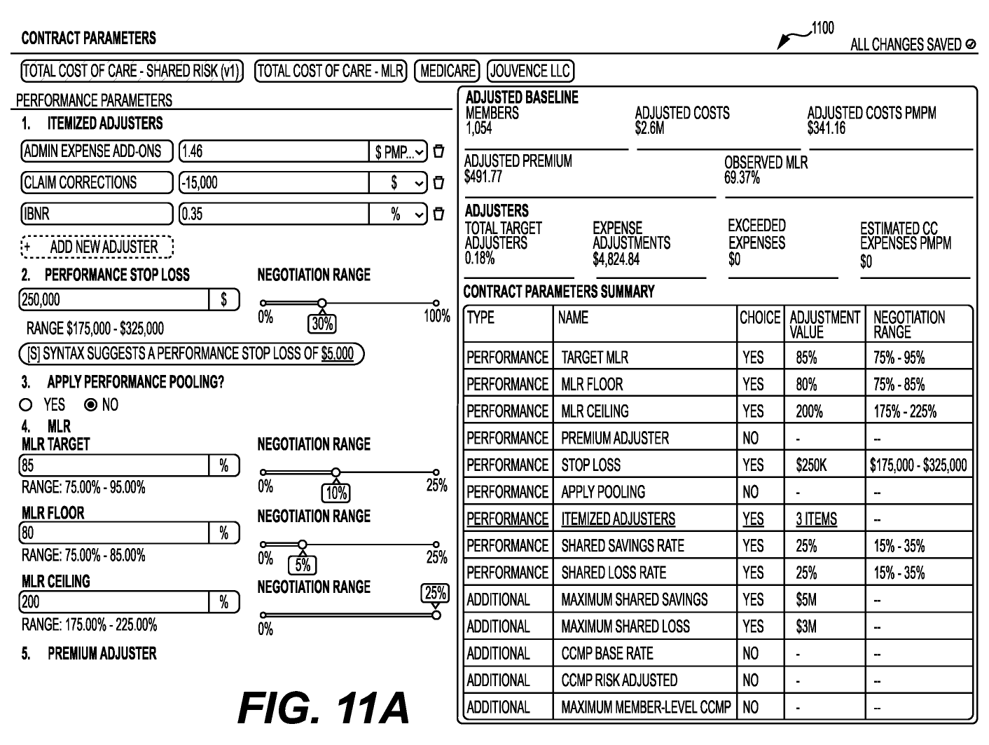

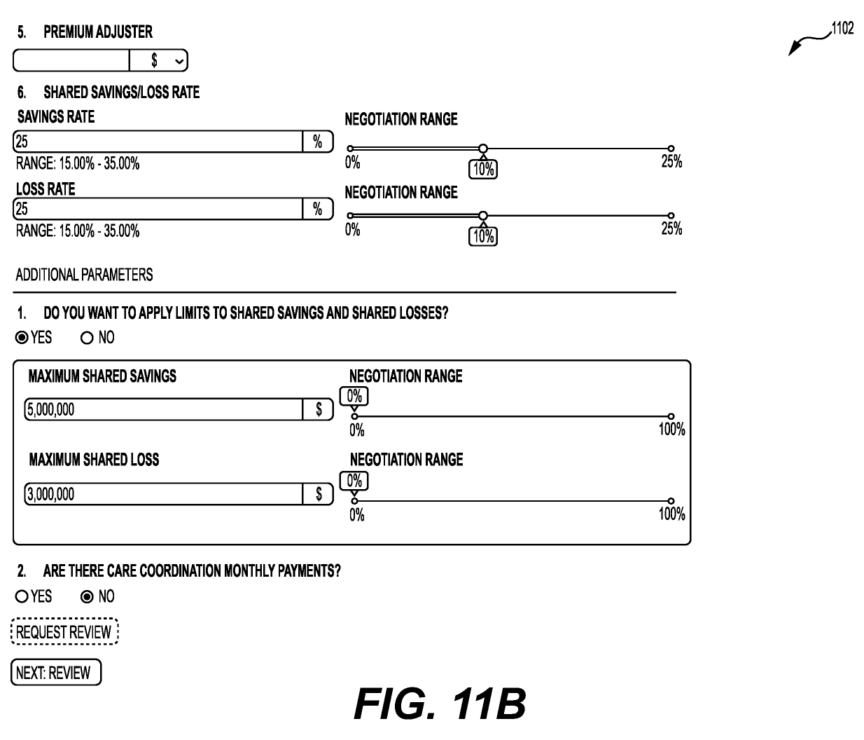

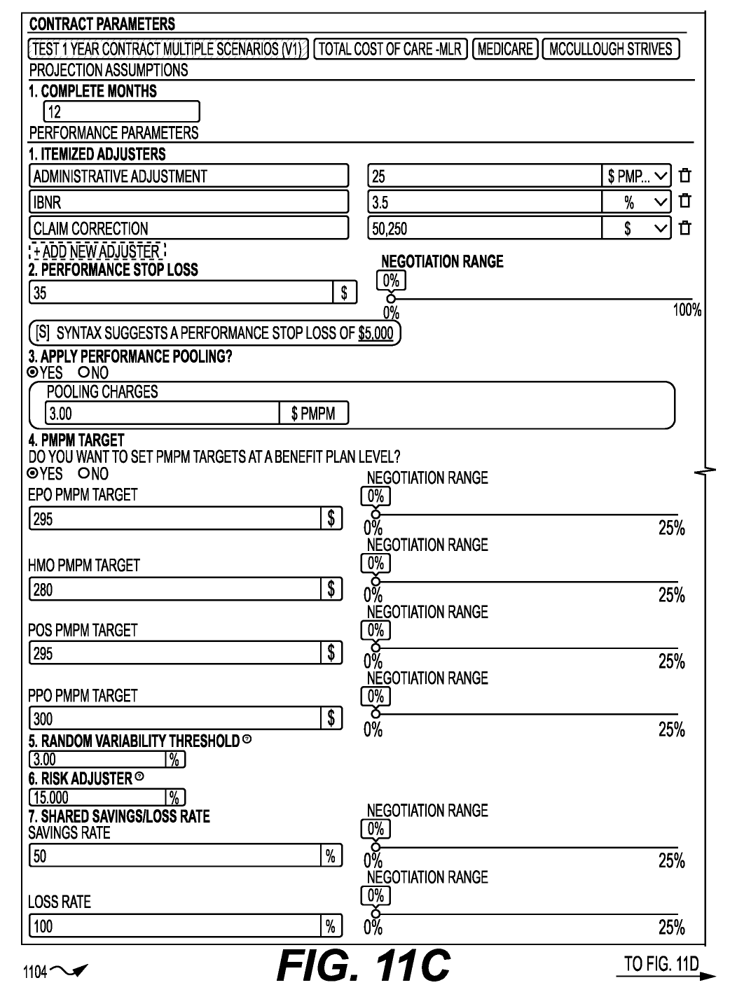

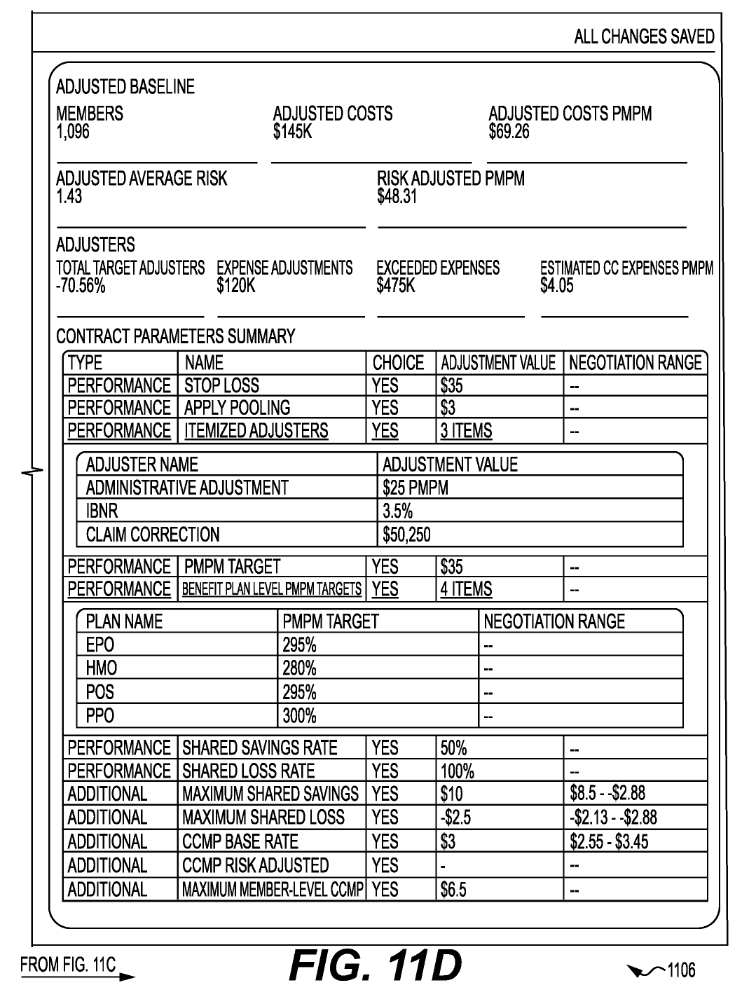

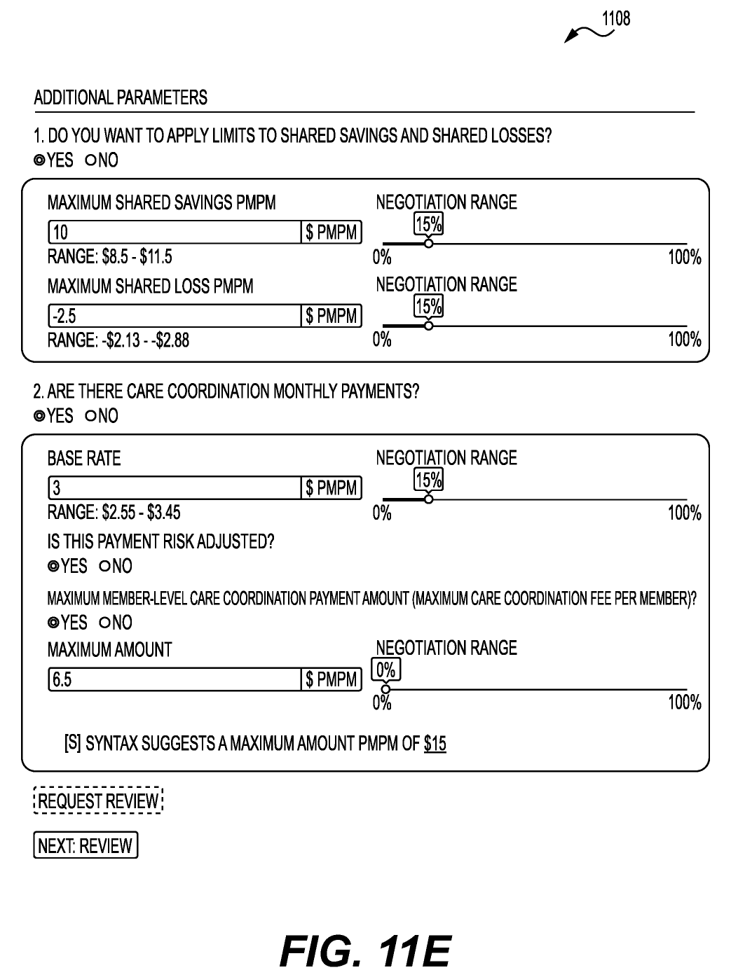

By implementing these parameters, contract parameters tool 414 may define the terms of VBC contracts as output of the contract builder tool. Contract parameters tool 414 may be further understood with respect to the example user screen of FIGS. 11A-11E.

Finally, enhanced VBC contract construction toolkit 400 may deliver information from contract parameters tool 414 to a contract review tool 316. Contract review tool 316 may utilize several sections to evaluate and summarize the terms and performance of VBC contracts. For a PMPM model, the sections may include attribution output, which may cover attributed lives, average risk score, and member months evaluated over a number of fiscal quarters. This section may help in understanding the member demographics and risk profiles attributed to the provider. The historic premium section may include gross premium, premium adjustment, and net premium, evaluated over a number of fiscal quarters, providing insights into the financial inflows under the PMPM model. Historic results under modeled terms may encompass paid expenses, stop loss exceeded expenses, excluded expenses, pooling charges, IBNR adjustment, and other expense adjustments, evaluated over a number of fiscal quarters, offering a detailed view of the financial outflows and adjustments. Savings/loss projection estimation may include contract parameters, parameter value, historic saving/loss calculation, project assumptions, and projected performance terms evaluated over a number of fiscal quarters, helping to forecast financial outcomes based on historical data and assumptions. Quality performance payment estimation may include composite level, result application, incentive type, incentive value, and parameter level, which may be crucial for determining the financial incentives tied to quality performance. Finally, the projected payable amount section may be based on contract parameters, parameter value, and incentive and projected earned amount, summarizing the expected financial obligations and earnings. In some cases, actuarial parameters may be dynamically selected and propagated through a selected model.

For an MLR model, the sections may include actuarial parameters, which may cover actual expenses trend, blending weights, inpatient IBNR, member benefit mix factor, outpatient IBNR, pharmacy IBNR, premium trend, risk adjuster, risk factor, and trending months, all evaluated over a number of fiscal periods (e.g., the annual basis over the last three years). This section may provide a comprehensive view of the actuarial assumptions and adjustments. The attribution output section may include attributed lives, average risk score, and member months evaluated over a number of fiscal quarters, similar to the PMPM model, to understand member demographics and risk profiles. Historic results under modeled terms may encompass paid expenses, stop loss exceeded expenses, excluded expenses, pooling charges, IBNR adjustment, and other expense adjustments, evaluated over a number of fiscal quarters, providing a detailed financial performance overview. Savings/loss projection estimation may include contract parameters, parameter value, historic saving/loss calculation, project assumptions, and projected performance terms evaluated over a number of fiscal quarters, offering financial forecasts based on historical data and assumptions. Quality performance payment estimation may include composite level, result application, incentive type, incentive value, and parameter level, essential for determining financial incentives tied to quality metrics. The projected payable amount section may be based on contract parameters, parameter value, and incentive and projected earned amount, summarizing the expected financial obligations and earnings.

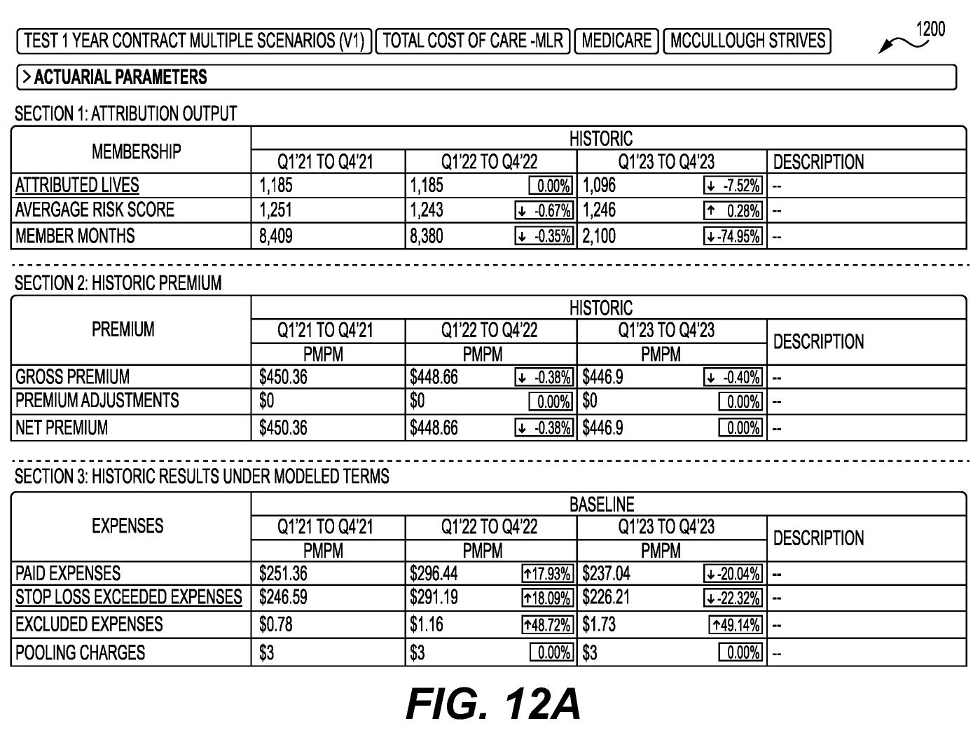

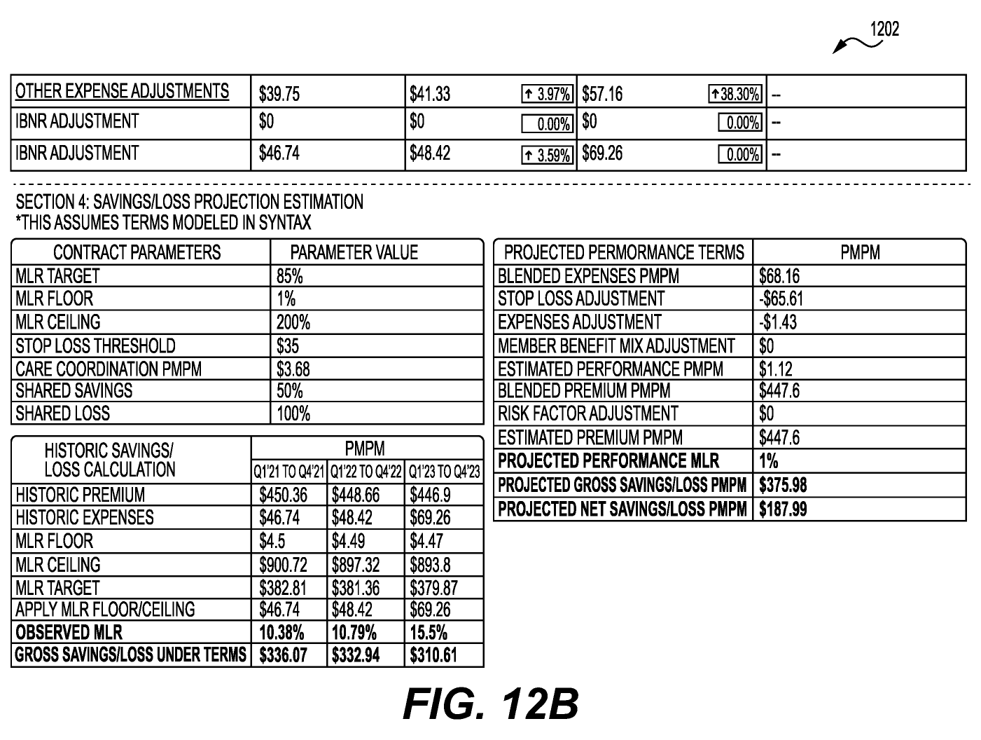

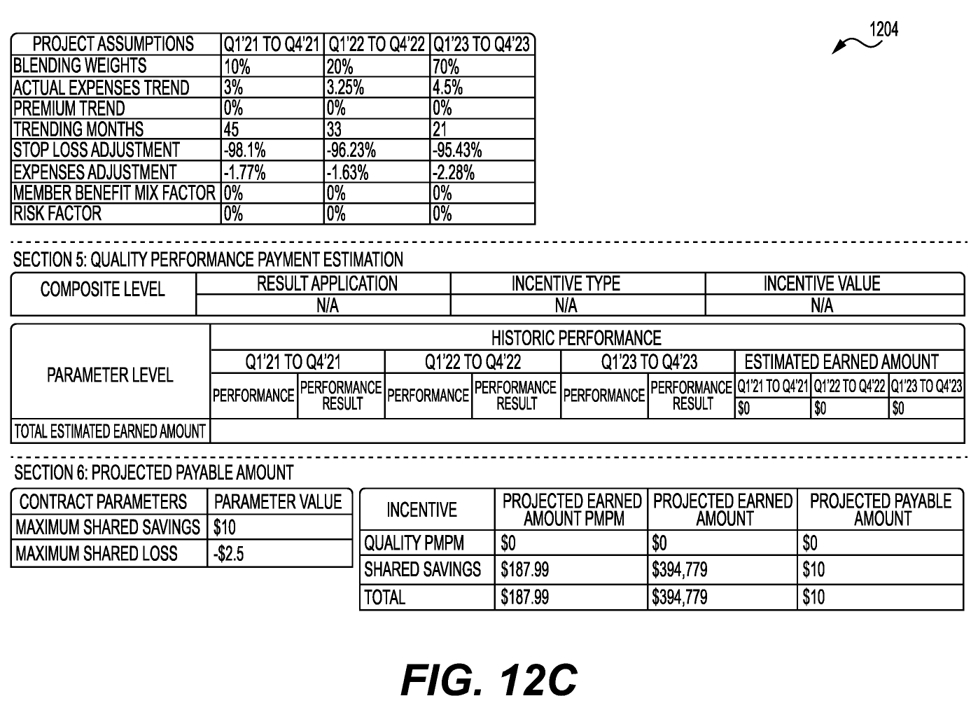

By implementing these sections—attribution output, historic premium, historic results under modeled terms, savings/loss projection estimation, quality performance payment estimation, and projected payable amount—contract review tool 316 may evaluate and summarize the terms and performance of VBC contracts. This comprehensive evaluation may help in understanding both the financial and quality performance aspects, ensuring that the contracts are aligned with the goals of value-based care. Contract review tool 316 may be further understood with respect to the example user screen of FIGS. 12A-12G.

### Example Toolkit for VBC Contract Construction

FIGS. 5-13 illustrate example user screens which may be displayed by a computer device at a user interface, where the computer device is communicatively coupled to an enhanced contract builder system configured to implement an enhanced VBC contract construction toolkit.

FIG. 5 illustrates an example screen 500 of a contract manager page displayed on a user interface. Screen 500 may provide functionalities including model, measure, manage, approvals, parameters, glossary, admin, and support. Users may define templates for attribution terms, cost terms, and quality parameters. The current contracts section may enable users to filter and compare contracts by various criteria. All contracts are in draft status, and there is an option to create a new contract.

FIG. 6A illustrates an example screen 600 of an affinity overview page displayed on a user interface. Screen 600 may indicate the number of individuals in a population who have utilized or selected a provider for services, identifying potentially attributable individuals. The total number of members may be 41,423, with an affinity assignment rate of 101.10 %. The top affinity types and provider specialties are detailed, along with visit counts.

FIG. 6B illustrates an example screen 602 of an affinity page displayed on a user interface. Screen 602 may allow users to filter by various criteria, including provider specialty and count of visits, and may provide options to export data.

FIG. 6C illustrates an example screen 604 of an affinity page selector displayed on a user interface. Screen 604 may allow users to view specific information for a given visit.

FIG. 7 illustrates an example screen 700 of an attribution page displayed on a user interface. Screen 700 may allow users to filter by various criteria, including attribution type, look-back period, and payer. The attribution type “Total Cost of Care MLR” may be highlighted, with options for both retrospective and prospective analysis. Users may select voluntary attribution as the first choice. The screen may also display information on payers and provider specialties. Exclusion criteria may encompass various conditions and high-cost members.

FIG. 8 illustrates an example screen 800 of a model selector page displayed on a user interface. Screen 800 may allow users to select from various model types for value-based payment design, including “Total Cost of Care - MLR,” “Total Cost of Care - Trend,” and “Total Cost of Care - PMPM.”

FIG. 9A illustrates an example screen 900 of a cost terms page displayed on a user interface. Screen 900 may allow users to manage performance attribution and cost exclusions for a test 1-year contract with multiple scenarios. The performance attribution type selected is “Total Cost of Care - MLR,” with a monthly performance period. Users can specify cost exclusions for both baseline and performance periods.

FIG. 9B illustrates an example screen 902 of a cost terms page displayed on a user interface. Screen 902 may allow users to manage baseline and performance attribution for a test 1-year contract with multiple scenarios. The total cost trend for the baseline period is set under the “Total Cost of Care - Trend” model. Users can specify cost exclusions for both baseline and performance periods.

FIG. 10 illustrates an example screen 1000 of a quality parameters page displayed on a user interface. Screen 1000 may allow users to manage quality parameters for the “Total Cost of Care - MLR” model. Users can specify whether to apply a composite quality result. Quality parameters may include various metrics, such as annual care visits, immunizations, readmissions, and follow-up within 10 days.

FIG. 11A illustrates an example screen 1100 of a contract parameters page displayed on a user interface. Screen 1100 may allow users to manage performance parameters for a contract under the “Total Cost of Care - Shared Risk” model. The performance parameters include itemized adjusters such as admin expense add-ons, claim corrections, and IBNR. Users can add new adjusters as needed.

FIG. 11B illustrates an example screen 1102 of a contract parameters page displayed on a user interface. Screen 1102 may allow users to manage premium adjusters and shared savings/loss rates for a contract. Users can specify additional parameters, including negotiation ranges for both savings and loss rates. Users can choose whether to apply limits to shared savings and shared losses.

FIG. 11C illustrates an example screen 1104 of a contract parameters page displayed on a user interface. Screen 1104 may allow users to manage projection assumptions and performance parameters for a contract under the “Total Cost of Care - Shared Risk” model. The projection assumptions include the completion of 12 months. The performance parameters include itemized adjusters such as administrative adjustments, IBNR, and claim corrections.

FIG. 11D illustrates an example screen 1106 of a contract parameters page displayed on a user interface. Screen 1106 may allow users to manage PMPM targets and other related parameters for a benefit plan. Users can choose to set PMPM targets at a benefit plan level.

FIG. 11E illustrates an example screen 1108 of a contract parameters page displayed on a user interface. Screen 1108 may allow users to manage additional parameters for a contract. Users can choose whether to apply limits to shared savings and shared losses. Users can also specify whether there are care coordination monthly payments.

FIG. 12A illustrates an example screen 1200 of a contract review page displayed on a user interface. Screen 1200 may allow users to manage actuarial parameters and review historic data for a contract. The screen is divided into several sections, including Attribution Output, Historic Premium, and Historic Results under Modeled Terms.

FIG. 12B illustrates an example screen 1202 of a contract review page displayed on a user interface. Screen 1202 may be continued from screen 1200 to show Section 4. Section 4:
Savings/Loss Projection Estimation includes various metrics such as contractual terms, historical data, MLR metrics, AR target values, and projected performance metrics.

FIG. 12C illustrates an example screen 1204 of a contract review page displayed on a user interface. Screen 1204 may be continued from screen 1202 to show Sections 5 and 6. Section 5:
Quality Performance Payment Estimation, which includes details on quality performance metrics. Section 6:
   Projected Payable Amount, which includes contract parameters and projected payable amounts.

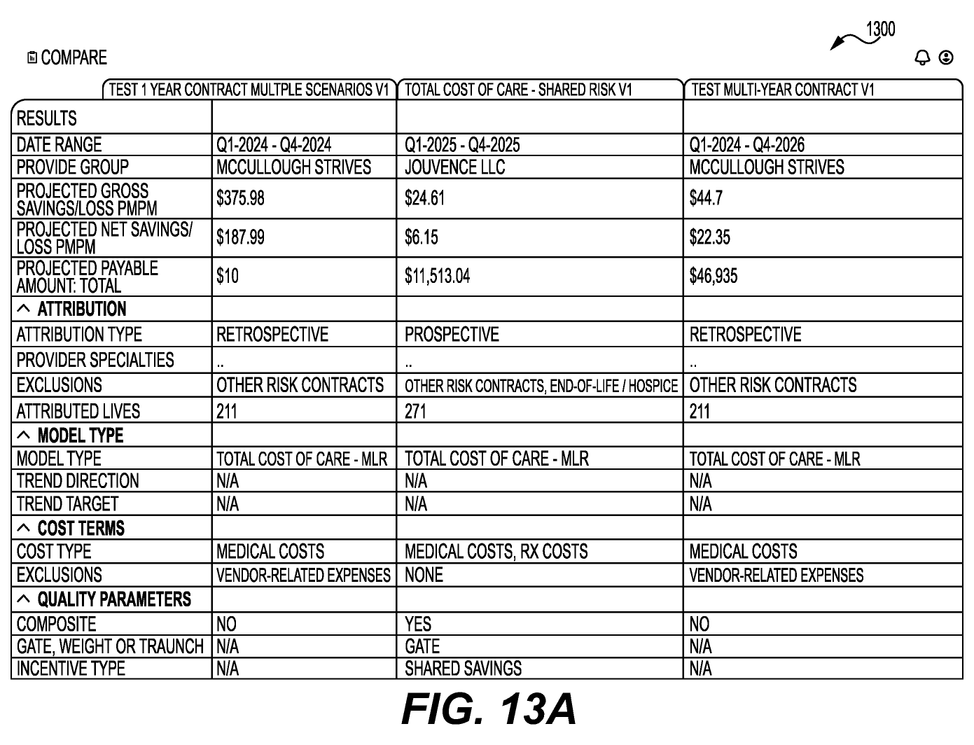

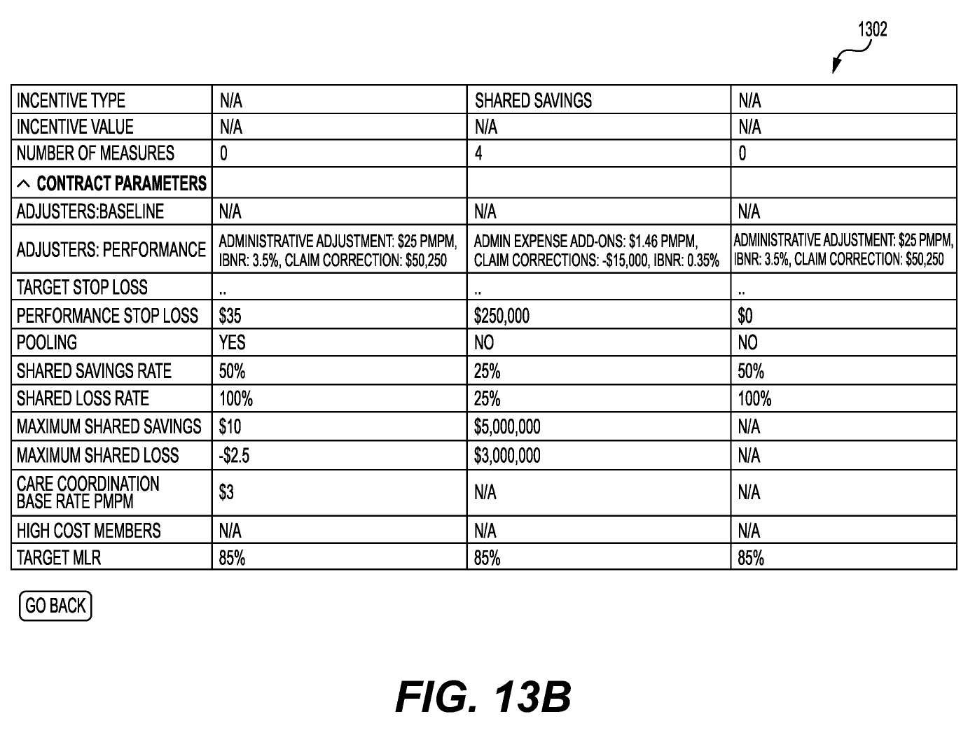

FIG. 13A illustrates an example screen 1300 of a contract review page displayed on a user interface. Screen 1300 may allow users to compare multiple scenarios for test contracts over different time periods. The results for the test 1-year contract with multiple scenarios and the test multi-year contract are displayed. The projected gross and net savings or loss per member per month (PMPM) and the projected payable amounts are detailed.

FIG. 13B illustrates an example screen 1302 of a contract review page displayed on a user interface. Screen 1302 may be continued from screen 1300 to show compared contract parameters. Contract parameters include various adjusters and performance metrics. Performance adjusters include administrative adjustments, IBNR, and claim corrections. The target stop loss and performance stop loss, pooling, shared savings rate, shared loss rate, maximum shared savings, maximum shared loss, care coordination base rate PMPM, and target MLR are detailed.

### EXAMPLE METHODS

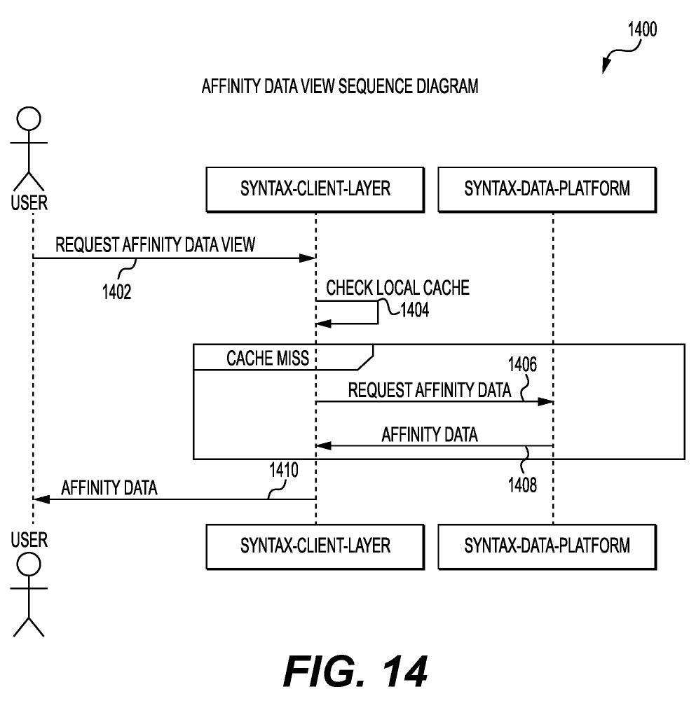

Each tool described above with respect to the in a toolkit for VBC contract construction (e.g., as described with respect to FIG. 3) may be accessed according to a call flow between a user device, a client layer, and a data platform (e.g., user interface 130, client layer 102, and data manager 110). By way of example, FIG. 14 illustrates a call flow of a method 1400 for accessing an affinity tool at a data platform via a client layer. Though the example of FIG. 14 is illustrated with respect to an affinity tool, method 1400 may be applicable to any tool, as suitable.

The method begins with a user sending a request 1402 for an affinity data view. The request 1402 may be initiated via a user device having a user interface. In one example, the user interface may display an option to select the affinity data view from a summary screen (e.g., screen 500 of FIG. 5). A client layer received the request 1402, and in response, the client layer performs a check 1404 of the local client layer cache. Where an affinity data view is available at the client layer local cache, the client layer may deliver the affinity data view to the user device and the method may be ended. Where an affinity data view is not available at the client layer local cache, the client layer proceeds to a cache miss sub-method by sending a request 1406 for affinity data to a data manager. In response to request 1406, the data manager may deliver a response 1408 with affinity data to the client layer. After receiving the response 1408, the client layer may send the affinity data to the user device in a response 1410. Once the affinity data is delivered to the user device, it may be displayed on a user interface for additional user interaction (e.g., via affinity screens described with respect to FIGS. 6A-C).

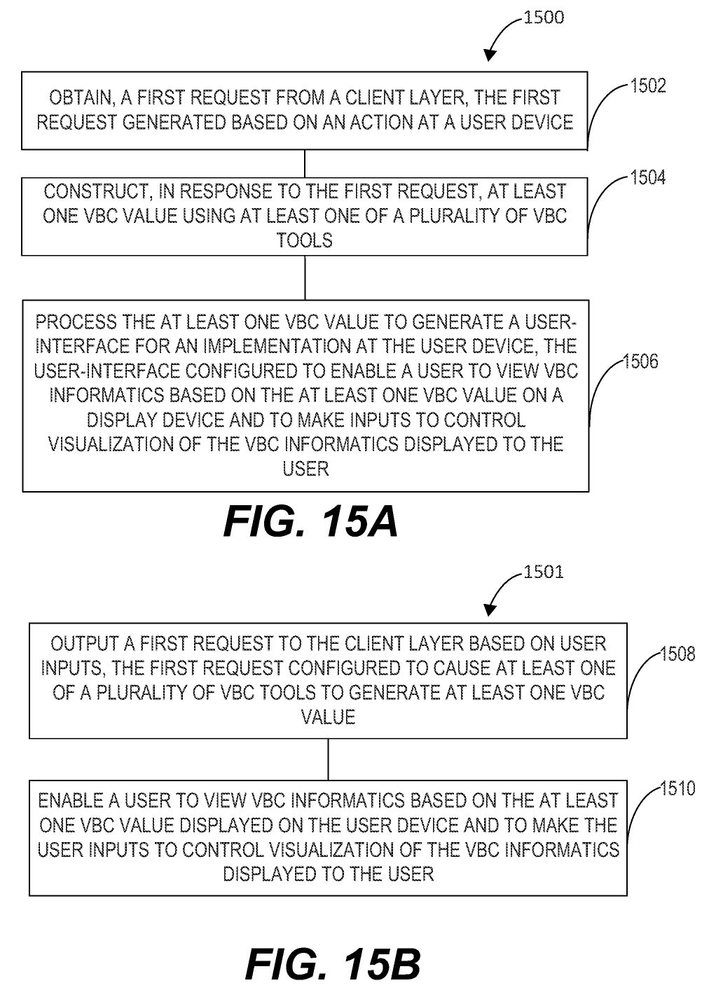

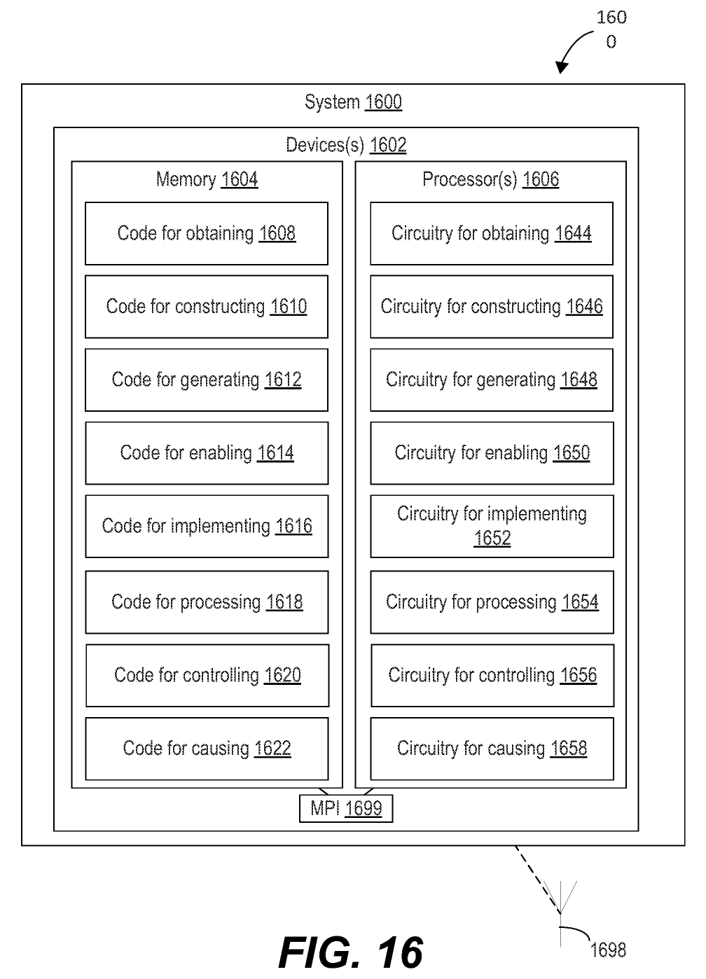

FIG. 15A is a schematic flowchart of an example method 1500 for VBC contract building by one or more processors, such as processors of enhanced contract builder system 100 and/or the processors of the system 1600 of FIG. 16. FIG. 15B is a schematic flowchart of an example method 1501 for VBC contract building by one or more processors, such as processors of enhanced contract builder system 100 and/or the processors of the system 1600 of FIG. 16.

Method 1500 begins at operation 1502 with a data manager implemented on a remote cloud service obtaining, a first request from a client layer, the first request generated based on an action at a user device.

Method 1500 continues to operation 1504 with the data manager constructing, in response to the first request, at least one VBC value using at least one of a plurality of VBC tools. In one example, the plurality of VBC tools includes an affinity tool configured to implement, while processing the at least one VBC value, at least one of a member identification, provider information, provider specialty information, affinity type information, a count of visits, visit span information, and last visit information. In one example, the plurality of VBC tools includes an attribution tool configured to implement, while processing the at least one VBC value, at least one of an attribution type, an attribution lookback, an attribution first choice, an attribution second choice, an attribution third choice, a tie breaker value, payer information, benefit plan information, provider specialty information, and exclusion criteria. In one example, the plurality of VBC tools includes a model type tool configured to implement, while processing the at least one VBC value, one of a medical loss ratio (MLR) model, a per member per month (PMPM) model, and a trend-based model. In one example, the plurality of VBC tools includes a cost terms tool configured to implement, while processing the at least one VBC value, baseline attribution information, a benchmark type, a baseline period, cost exclusions, performance attribution information, a performance period, and a performance cost. In one example, the plurality of VBC tools includes a quality parameters tool configured to implement, while processing the at least one VBC value, composite information, quality result application information, and quality performance incentive type. In one example, the plurality of VBC tools includes a choice of provider tool. In one example, the plurality of VBC tools includes a contract parameters tool configured to allow the user to perform itemized adjustments for one or more parameters, the itemized adjustments propagated to the at least one VBC value via the data manager. In one example, the itemized adjustments include at least one of:
administrative expense add-ons, claim corrections, incurred but not reported (IBNR) claim information, medical loss ratio (MLR) target information, and a shared savings/loss rate. In one example, the itemized adjustments include at least one of:
   complete month information, administrative adjustment information, PMPM target information, and incurred but not reported (IBNR) claim information. In one example, the data manager is configured to facilitate implementing the plurality of VBC tools for multiple requests from a plurality of user devices.

Method 1500 optionally continues to operation 1506 with a client layer processing the at least one VBC value to generate a user-interface for an implementation at the user device, the user-interface configured to enable a user to view VBC informatics based on the at least one VBC value on a display device and to make inputs to control visualization of the VBC informatics displayed to the user. In one example, the inputs are dynamic actuarial inputs configured to generate the at least one VBC value.

In one aspect, method 1500, or any aspect related to it, may be performed by a system, device, apparatus, or architecture, such as enhanced contract builder system 100 or system 1600 of FIG. 16, which includes various components operable, configured to, or adapted to perform the method 1500. System 1600 is described below in further detail.

FIG. 15A is just one example of a method, and other methods including fewer, additional, or alternative operations are contemplated consistent with the disclosure.

Method 1501 begins at operation 1508 with a user device communicatively coupled to a client layer over a network outputting a first request to the client layer based on user inputs, the first request configured to cause at least one of a plurality of VBC tools to generate at least one VBC value. In one example, the plurality of VBC tools includes an affinity tool configured to implement, while processing the at least one VBC value, at least one of a member identification, provider information, provider specialty information, affinity type information, a count of visits, visit span information, and last visit information. In one example, the plurality of VBC tools includes an attribution tool configured to implement, while processing the at least one VBC value, at least one of an attribution type, an attribution lookback, an attribution first choice, an attribution second choice, an attribution third choice, a tie breaker value, payer information, benefit plan information, provider specialty information, and exclusion criteria. In one example, the plurality of VBC tools includes a model type tool configured to implement, while processing the at least one VBC value, one of a MLR model, a PMPM model, and a trend-based model. In one example, the plurality of VBC tools includes a cost terms tool configured to implement, while processing the at least one VBC value, baseline attribution information, a benchmark type, a baseline period, cost exclusions, performance attribution information, a performance period, and a performance cost. In one example, the plurality of VBC tools includes a quality parameters tool configured to implement, while processing the at least one VBC value, composite information, quality result application information, and quality performance incentive type. In one example, the plurality of VBC tools includes a contract parameters tool configured to allow the user to perform itemized adjustments for one or more parameters, the itemized adjustments propagated to the at least one VBC value via a data manager.

Method 1501 continues to operation 1510 with a user-interface displayed on the user device enabling a user to view VBC informatics based on the at least one VBC value displayed on the user device and to make the user inputs to control visualization of the VBC informatics displayed to the user. In one example, the user inputs are dynamic actuarial inputs configured to generate the at least one VBC value.

In one aspect, method 1501, or any aspect related to it, may be performed by a system, device, apparatus, or architecture, such as enhanced contract builder system 100 or system 1600 of FIG. 6, which includes various components operable, configured to, or adapted to perform the method 1501. System 1600 is described below in further detail.

FIG. 15B is just one example of a method, and other methods including fewer, additional, or alternative operations are contemplated consistent with the disclosure.

### EXAMPLE SYSTEM

FIG. 16 illustrates a schematic diagram of an example system 1600, which includes a device 1602. The system 1600 may be implemented as part of the procedures described with respect to FIGS. 1-15B. Device(s) 1602 may be implemented at a single location or at multiple locations and may be a supervisory unit or may be in communication with a supervisory unit by way of a communication line. In at least one embodiment, the communication line may be a wireless communication line, a wired communication line, or both, though other types of communication line are contemplated. Device(s) 1602 may include any of a user device, a client layer, a data manager, or any other suitable component (e.g., any component of system 100).

The device(s) 1602 may include a CPU processing system, which may be configured to perform enhanced LUP procedures, such as those described herein with respect to FIG. 1-15B, as performed by the system 1600. The CPU processing system of the device(s) 1602 may include one or more processors 1606 coupled to a computer readable medium/memory 1604, for example via a bus (not shown). The one or more processors 1606 and the computer readable medium/memory 1604 may communicate, for example, via a message passing interface (MPI) 1636. In certain aspects the computer readable medium/ memory 1604 is configured to store instructions (e.g., computer executable code) that when executed by the one or more processors 1606, cause the one or more processors to perform the method 1500 described with respect to FIGS. 15A-15B, or any aspect related to it. Reference to a single processor performing a function of system 1600 may include one or more processors performing that function of system 1600.

In the depicted example, computer-readable medium/memory 1604 stores code 1608 (e.g., executable instructions) for obtaining, code 1610 for constructing, code 1612 for generating, code 1614 for enabling, code 1616 for implementing, code 1618 for processing, code 1620 for controlling, and/or code 1622 for causing. Processing of code 1608-1622 may cause the system 1600 to perform the method 1500 described with respect to FIGS. 15A-15B, or any aspect related to it.

The one or more processors 1606 include circuitry configured to implement (e.g., execute) the code stored in the computer-readable medium / memory 1604, including circuitry 1644 for obtaining, circuitry 1646 for constructing, circuitry 1648 for generating, circuitry 1650 for enabling, circuitry 1652 for implementing, circuitry 1654 for processing, circuitry 1656 for controlling, and/or circuitry 1658 for causing. Processing with circuitry 1644-1658 may cause the system 1600 to perform the method 1500 described with respect to FIGS. 15A-15B, or any aspect related to it.

Various components of the system 1600 may provide means for performing the method 1500 described with respect to FIGS. 15A-15B, or any aspect related to it.

The system 1600 may include or be substantially coupled to a communication component 1642. In the depicted example, the communication component 1642 is an antenna capable of communicating with controllers similar to system 1600 to perform the method 1500 described with respect to FIGS. 15A-15B, or any aspect related to it. In additional examples, the communication component 1642 may be a bus or a wired connection.

### EXAMPLE COMPUTER SYSTEM

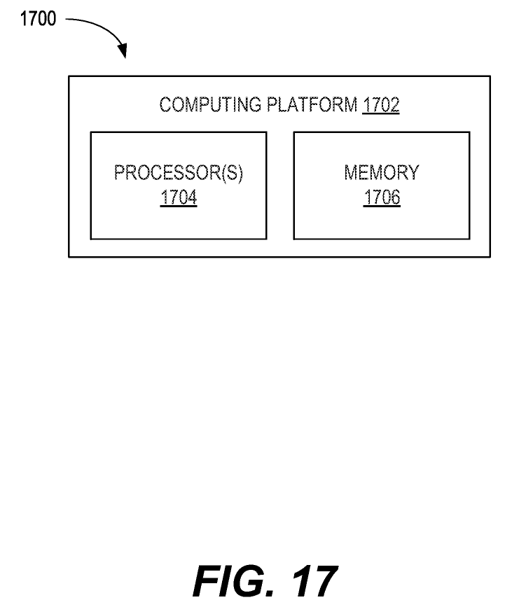

FIG. 17 is an example of a block diagram of a system 1700. System 1700 can be implemented using one or more modules, shown in block form in the drawings. The one or more modules can be in software or hardware form, or a combination thereof. In some examples, the system 1700 can be implemented as machine readable instructions for execution on one or more computing platforms 1702 (referred to as a computing platform herein), as shown in FIG. 17. The computing platform 1702 can include one or more computing devices selected from, for example, a desktop computer, a server, a controller, a blade, a mobile phone, a tablet, a laptop, a personal digital assistant (PDA), and the like.

The computing platform 1704 can include a processor 1704 and a memory 1706. By way of example, the memory 1706 can be implemented, for example, as a non-transitory computer storage medium, such as volatile memory (e.g., random access memory), non-volatile memory (e.g., a hard disk drive, a solid-state drive, a flash memory, or the like), or a combination thereof. The processor 1704 can be implemented, for example, as one or more processor cores. The memory 1706 can store machine-readable instructions that can be retrieved and executed by the processor 1704 to implement the system 1700. Each of the processor 1704 and the memory 1706 can be implemented on a similar or a different computing platform. The computing platform 1702 can be implemented in a cloud computing environment (for example, as disclosed herein) and thus on a cloud infrastructure. In such a situation, features of the computing platform 1702 can be representative of a single instance of hardware or multiple instances of hardware executing across the multiple of instances (e.g., distributed) of hardware (e.g., computers, routers, memory, processors, or a combination thereof). Alternatively, the computing platform 1702 can be implemented on a single dedicated server or workstation.

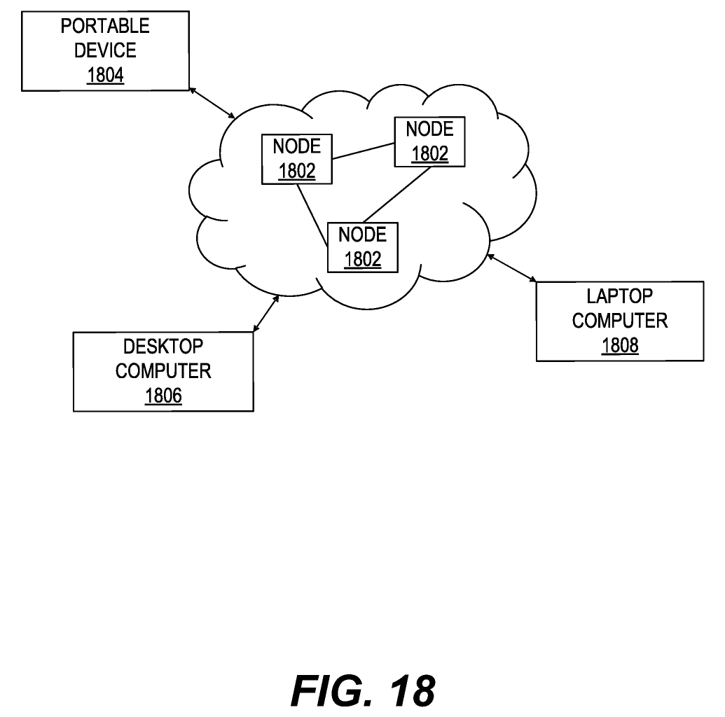

FIG. 18 is an example of a cloud computing environment 1800 that can be used for implementing one or more modules and/or systems in accordance with one or more examples, as disclosed herein. As shown, cloud computing environment 1800 can include one or more cloud computing nodes 1802 with which local computing devices used by cloud consumers (or users), such as, for example, personal digital assistant (PDA), cellular, or portable device 1804, a desktop computer 1806, and/or a laptop computer 1808, may communicate. The computing nodes 1802 can communicate with one another. In some examples, the computing nodes 1802 can be grouped (not shown) physically or virtually, in one or more networks, such as Private, Community, Public, or Hybrid clouds, or a combination thereof. This allows the cloud computing environment 1800 to offer infrastructure, platforms and/or software as services for which a cloud consumer does not need to maintain resources on a local computing device. The devices 1804-1808, as shown in FIG. 18, are intended to be illustrative and that computing nodes 1802 and cloud computing environment 1800 can communicate with any type of computerized device over any type of network and/or network addressable connection (e.g., using a web browser). In some examples, the one or more computing nodes 1802 are used for implementing one or more examples disclosed herein relating to root-source identification. Thus, in some examples, the one or more computing nodes can be used to implement modules, platforms, and/or systems, as disclosed herein.

In some examples, the cloud computing environment 1800 can provide one or more functional abstraction layers. It is to be understood that the cloud computing environment 1800 need not provide all of the one or more functional abstraction layers (and corresponding functions and/or components), as disclosed herein. For example, the cloud computing environment 1800 can provide a hardware and software layer that can include hardware and software components. Examples of hardware components include:
mainframes; RISC (Reduced Instruction Set Computer) architecture based servers;
servers; blade servers; storage devices; and
networks and networking components. In some embodiments, software components include network application server software and database software.

In some examples, the cloud computing environment 1800 can provide a virtualization layer that provides an abstraction layer from which the following examples of virtual entities may be provided:
virtual servers;
virtual storage;
virtual networks, including virtual private networks;
virtual applications and operating systems; and
virtual clients. In some examples, the cloud computing environment 1800 can provide a management layer that can provide the functions described below. For example, the management layer can provide resource provisioning that can provide dynamic procurement of computing resources and other resources that are utilized to perform tasks within the cloud computing environment. The management layer can also provide metering and pricing to provide cost tracking as resources are utilized within the cloud computing environment 1800, and billing or invoicing for consumption of these resources. In one example, these resources may include application software licenses. Security provides identity verification for cloud consumers and tasks, as well as protection for data and other resources. The management layer can also provide a user portal that provides access to the cloud computing environment 1800 for consumers and system administrators. The management layer can also provide service level management, which can provide cloud computing resource allocation and management such that required service levels are met. Service Level Agreement (SLA) planning and fulfillment can also be provided to provide pre-arrangement for, and procurement of, cloud computing resources for which a future requirement is anticipated in accordance with an SLA.

In some examples, the cloud computing environment 1800 can provide a workloads layer that provides examples of functionality for which the cloud computing environment 1800 may be utilized. Examples of workloads and functions which may be provided from this layer include:
mapping and navigation;
software development and lifecycle management;
virtual classroom education delivery;
data analytics processing; and
transaction processing. Various embodiments of the present disclosure can utilize the cloud computing environment 1800.

### EXAMPLE ASPECTS

Implementation examples are described in the following numbered clauses:

Aspect 1: A system for value-based care (VBC) contract construction, including:
a data manager implemented on a remote cloud service and configured to:
   obtain, a first request from a client layer, the first request generated based on an action at a user device; and
   construct, in response to the first request, at least one VBC value using at least one of a plurality of VBC tools; and
   the client layer communicatively coupled to the data manager and configured to process the at least one VBC value to generate a user-interface for an implementation at the user device, the user-interface configured to enable a user to view VBC informatics based on the at least one VBC value on a display device and to make inputs to control visualization of the VBC informatics displayed to the user.

Aspect 2: The system of aspect 1, wherein the plurality of VBC tools includes an affinity tool configured to implement, while processing the at least one VBC value, at least one of a member identification, provider information, provider specialty information, affinity type information, a count of visits, visit span information, and last visit information.

Aspect 3: The system of any one of aspects 1 through 2, wherein the plurality of VBC tools includes an attribution tool configured to implement, while processing the at least one VBC value, at least one of an attribution type, an attribution lookback, an attribution first choice, an attribution second choice, an attribution third choice, a tie breaker value, payer information, benefit plan information, provider specialty information, and exclusion criteria.

Aspect 4: The system of any one of aspects 1 through 3, wherein the plurality of VBC tools includes a model type tool configured to implement, while processing the at least one VBC value, one of a medical loss ratio (MLR) model, a per member per month (PMPM) model, and a trend-based model.

Aspect 5: The system of any one of aspects 1 through 4, wherein the plurality of VBC tools includes a cost terms tool configured to implement, while processing the at least one VBC value, baseline attribution information, a benchmark type, a baseline period, cost exclusions, performance attribution information, a performance period, and a performance cost.

Aspect 6: The system of any one of aspects 1 through 5, wherein the plurality of VBC tools includes a quality parameters tool configured to implement, while processing the at least one VBC value, composite information, quality result application information, and quality performance incentive type.

Aspect 7: The system of any one of aspects 1 through 6, wherein the plurality of VBC tools includes a choose a provider tool.

Aspect 8: The system of any one of aspects 1 through 7, wherein the plurality of VBC tools includes a contract parameters tool configured to allow the user to perform itemized adjustments for one or more parameters, the itemized adjustments propagated to the at least one VBC value via the data manager.

Aspect 9: The system of aspect 8, wherein the one or more parameters include at least one of:
administrative expense add-ons, claim corrections, incurred but not reported (IBNR) claim information, medical loss ratio (MLR) target information, and a shared savings/loss rate.

Aspect 10: The system of any one of aspects 8 through 9, wherein the one or more parameters include at least one of:
complete month information, administrative adjustment information, PMPM target information, and incurred but not reported (IBNR) claim information.

Aspect 11: The system of any one of aspects 1 through 10, wherein the inputs are dynamic actuarial inputs configured to generate the at least one VBC value.

Aspect 12: The system of any one of aspects 1 through 11, wherein the data manager is configured to facilitate implementing the plurality of VBC tools for multiple requests from a plurality of user devices.

Aspect 13: A system for VBC contract construction, including:
a user device communicatively coupled to a client layer over a network and configured to output a first request to the client layer based on user inputs, the first request configured to cause at least one of a plurality of VBC tools to generate at least one VBC value; and
a user-interface displayed on the user device, the user-interface configured to enable a user to view VBC informatics based on the at least one VBC value displayed on the user device and to make the user inputs to control visualization of the VBC informatics displayed to the user.

Aspect 14: The system of aspect 13, wherein the plurality of VBC tools includes an affinity tool configured to implement, while processing the at least one VBC value, at least one of a member identification, provider information, provider specialty information, affinity type information, a count of visits, visit span information, and last visit information.

Aspect 15: The system of any one of aspects 13 through 14, wherein the plurality of VBC tools includes an attribution tool configured to implement, while processing the at least one VBC value, at least one of an attribution type, an attribution lookback, an attribution first choice, an attribution second choice, an attribution third choice, a tie breaker value, payer information, benefit plan information, provider specialty information, and exclusion criteria.

Aspect 16: The system of any one of aspects 13 through 15, wherein the plurality of VBC tools includes a model type tool configured to implement, while processing the at least one VBC value, one of a medical loss ratio (MLR) model, a per member per month (PMPM) model, and a trend-based model.

Aspect 17: The system of any one of aspects 13 through 16, wherein the plurality of VBC tools includes a cost terms tool configured to implement, while processing the at least one VBC value, baseline attribution information, a benchmark type, a baseline period, cost exclusions, performance attribution information, a performance period, and a performance cost.

Aspect 18: The system of any one of aspects 13 through 17, wherein the plurality of VBC tools includes a quality parameters tool configured to implement, while processing the at least one VBC value, composite information, quality result application information, and quality performance incentive type.

Aspect 19: The system of any one of aspects 13 through 18, wherein the plurality of VBC tools includes a contract parameters tool configured to allow the user to perform itemized adjustments for one or more parameters, the itemized adjustments propagated to the at least one VBC value via a data manager.

Aspect 20: The system of any one of aspects 13 through 19, wherein the user inputs are dynamic actuarial inputs configured to generate the at least one VBC value.

Aspect 21: An apparatus or device including a memory comprising executable instructions, and a processor configured to execute the executable instructions and cause the apparatus to perform a method in accordance with any one of aspects 1-20.

Aspect 22: An apparatus or device, including means for performing a method in accordance with any one of aspects 1-20.

Aspect 23: A non-transitory computer-readable medium including executable instructions that, when executed by a processor of an apparatus, cause the apparatus to perform a method in accordance with any one of aspects 1-20.

Aspect 24: A computer program product embodied on a computer-readable storage medium comprising code for performing a method in accordance with any one of aspects 1-20.

### EXAMPLE THREAT MODEL

In at least one aspect, data may be processed via a data unit (e.g., primary data unit 110) to perform enhanced value-based care (VBC) contract procedures at the enhanced VBC architecture. The following describes each data type with respect to their information security rating. In information security, confidentiality impact rating (C) protects information from unauthorized access; integrity impact rating (I) ensures data accuracy and reliability; and availability impact rating (A) ensures information and resources are accessible to authorized users when needed.

Member Demographics:
The type of data represented by this asset is protected health information (PHI). This asset may include member demographic data such as name, date of birth, sex at birth, geographic information, and other information associated with a member. The impacts to confidentiality, integrity, and availability in the event of a breach could be as follows:
   C: Any breach of this information might constitute a Health Insurance Portability and Accountability Act (HIPAA) breach.
   I: Altering this data could impact the accuracy of projections;
   for example, auto-assignment based on geography might not work. This asset may be persisted in primary data unit 110.

Encounter Data:
The type of data represented by this asset is PHI. This asset may include information about encounters such as diagnosis codes, procedures, rendering national provider identifiers (NPIs), and other related data. The impacts to confidentiality, integrity, and availability in the event of a breach could be as follows:
   C: Any breach of this information might constitute a HIPAA breach.
   I: Altering this data could impact the accuracy of projections;
      for example, members could be erroneously excluded due to incorrect diagnosis codes or incorrect NPIs. This asset may be persisted in primary data unit 110.

Claims Data:
The type of data represented by this asset is PHI. This asset may include claim headers and claim lines, encompassing medical, pharmacy, and lab claims. The impacts to confidentiality, integrity, and availability in the event of a breach could be as follows:
   C: Any breach of this information might constitute a HIPAA breach.
   I: Altering this data could impact the accuracy of projections. This asset may be persisted in primary data unit 110.

Enrollment Data:
The type of data represented by this asset is PHI. This asset may include information about a member's enrollment with an insurance plan, including effective dates, group name, product code, and Primary Care Provider (PCP) information. The impacts to confidentiality, integrity, and availability in the event of a breach could be as follows:
   C: A breach of this information might constitute a HIPAA breach.
   I: Altering this data could impact the accuracy of projections;
   for example, member months might be calculated incorrectly. This asset may be persisted in primary data unit 110.

Premium Data:
The type of data represented by this asset includes PHI and business secrets. This asset may include information about the premiums associated with a member's coverage, including prescription (RX) and medical premiums. The impacts to confidentiality, integrity, and availability in the event of a breach could be as follows:
   C: A breach of this information in conjunction with dates or any member identification (ID) might constitute a HIPAA breach.
   C: Information about premium amounts, even without accompanying PHI, is confidential business information. This asset may be persisted in primary data unit 110.

Risk Score:
The type of data represented by this asset is PHI. This asset may include the risk score associated with a given member in a particular month. The impacts to confidentiality, integrity, and availability in the event of a breach could be as follows:
   C: A breach of this information might constitute a HIPAA breach.
   I: Altering this data could impact the accuracy of projections. This asset may be persisted in primary data unit 110.

Provider Demographics:
The type of data represented by this asset is personally identifiable information (PII). This asset may include demographics around healthcare providers such as name, geographic data, parent organization, and NPIs. The impacts to confidentiality, integrity, and availability in the event of a breach could be as follows:
   C: A breach of this data could reveal business secrets.
   I: Altering this data could impact the accuracy of projections. This asset may be persisted in primary data unit 110.

User Info:
The type of data represented by this asset is PII. This asset may include information about users such as email addresses and names. The impacts to confidentiality, integrity, and availability in the event of a breach could be as follows:
   C: An attacker with access to a user's personal data could leak the data publicly and expose sensitive user information.
   A: Modifying a user’s information, for example changing their email, could prevent that user from accessing the system. This asset may be persisted in database 108.

Contract Info:
The type of data represented by this asset is Business Secrets. This asset may include information about value-based care contracts, including contracting parties, contract terms, and contract parameters. The impacts to confidentiality, integrity, and availability in the event of a breach could be as follows:
   C: Exposing this information could cause material harm to client organizations because this is confidential business information.
   I: Altering this data could negatively impact payer-provider organizations.
   I: Altering this data after a user has reviewed the contract but before the contract has been printed could result in an individual signing a contract that is different from what they had reviewed. This asset may be persisted in database 108.

Provider Contracting Entities:
The type of data represented by this asset is Business Secrets. This asset may include information about provider contracting entities such as provider group taxpayer identification numbers (TINs), grouping of TINs, and groupings of NPIs. The impacts to confidentiality, integrity, and availability in the event of a breach could be as follows:
   C: A breach of this data could reveal business secrets.
   I: Altering this data could impact the accuracy of projections. This asset may be persisted in database 108.

Contract Chat Channel:
The types of data represented by this asset include PII, PHI, and Business Secrets. This asset is a chat channel where payor, provider, and payor/provider chat can facilitate contract creation. The impacts to confidentiality, integrity, and availability in the event of a breach could be as follows:
   C: Any breach of this information might constitute a HIPAA breach.
   C: A breach of this data could reveal business secrets.
   I: Altering this data could impact payor/provider relationships and cause the users of the system to lose faith. This asset may be persisted in chat manager 126.

Admin Login Credentials:
This asset includes the credentials for an admin to access the admin console and the application itself. The impacts to confidentiality, integrity, and availability in the event of a breach could be as follows:
   C: An attacker could view PII associated with users, PHI associated with members via the Affinity function, provider information, and confidential business information such as VBC contracts.
   I: An attacker could manipulate contract terms or parameters. This asset may be persisted in User Member Demographics, Encounter Data, Provider Demographics, User Info, Contract Info, and Provider Contracting Entities.

User Login Credentials:
This asset includes the credentials for a user to access the application. The impacts to confidentiality, integrity, and availability in the event of a breach could be as follows:
   C: An attacker could view PHI associated with members via the Affinity function, provider information, and confidential business information such as VBC contracts.
   I: An attacker could create and manipulate contract terms or parameters. This asset may be persisted in User Member Demographics, Encounter Data, Provider Demographics, User Info, Contract Info, and Provider Contracting Entities.

Terraform AWS Credentials:
This asset includes the AWS credentials used to provision and change AWS infrastructure. The impacts to confidentiality, integrity, and availability in the event of a breach could be as follows:
   C: An attacker could alter all settings on the infrastructure, threatening the confidentiality of assets.
   I: An attacker could alter all settings on the infrastructure, threatening the integrity of assets.
   A: An attacker could alter all settings on the infrastructure, threatening the availability of assets. This asset may be persisted in GitHub Secrets.

GitHub Credentials:
This asset includes the credentials for accessing GitHub. Currently, only members or third-party development partners have access to the source code. The impacts to confidentiality, integrity, and availability in the event of a breach could be as follows:
   C: An attacker could modify the source code to compromise user data confidentiality and generate a GitHub Personal Access Token to access Vault.
   I: An attacker could modify the source code and inject malicious or defective code.
   A: An attacker could make the service non-functional and prevent code fixes from being deployed. This asset may be persisted in User All.

GitHub Actions AWS API Credentials:
This asset includes the AWS credentials used by the client layer to perform its functions within AWS. The impacts to confidentiality, integrity, and availability in the event of a breach could be as follows:
   C: An attacker could access data housed in primary data unit 110 and the client-layer-database directly.
   I: An attacker could mutate data in both primary data unit 110 and the client-layer-database, change user settings stored in Vault, and impersonate the email.
   A: An attacker obtaining access to AWS credentials could change infrastructure and access permissions or user credentials, rendering the system unavailable. This asset may be persisted in GitHub Secrets and environment variables.

Postgres Credentials:
This asset includes the credentials used to access the client-layer-database. The impacts to confidentiality, integrity, and availability in the event of a breach could be as follows:
   C: An attacker in possession of the Postgres Credentials could access sensitive information stored in the client-layer-database.
   I: An attacker in possession of the Postgres Credentials could mutate sensitive information stored in the client-layer-database. This asset may be persisted in Vault, AWS Secrets Manager, and environment variables. The functional assets affected include User Info, Contract Info, and Provider Contracting Entities.

Redshift Credentials:
This asset includes the credentials used to access primary data unit 110. The impacts to confidentiality, integrity, and availability in the event of a breach could be as follows:
   C: An attacker in possession of the Redshift Credentials could access sensitive information stored in primary data unit 110.
   I: An attacker in possession of the Redshift Credentials could mutate sensitive information stored in primary data unit 110. This asset may be persisted in Vault, AWS Secrets Manager, and environment variables. The functional assets affected include Member Demographics, Encounter Data, Claims Data, Enrollment Data, Premium Data, Risk Score, and Provider Demographics.

Datadog API Credentials:
This asset includes the credentials used to push logging and telemetry data to Datadog. Writing PII/PHI to logs is discouraged but not strictly prohibited. Therefore, access to Datadog logs could theoretically threaten PHI. The impacts to confidentiality, integrity, and availability in the event of a breach could be as follows:
   C: An attacker could read privileged log entries and gain a better understanding of system vulnerabilities.
   I: An attacker could post erroneous logs. This asset may be persisted in Vault, GitHub Secrets, and environment variables. There are no functional assets directly affected, but the details in the description highlight potential indirect impacts.

DBT Credentials:
This asset includes the credentials with which a user may log into DBT. The impacts to confidentiality, integrity, and availability in the event of a breach could be as follows:
   C: An attacker could run a DBT job that outputs sensitive data to DBT logs, compromising data confidentiality.
   I: An attacker could execute a DBT job that modifies data, impacting data integrity. This asset may be persisted on engineers' machines. The functional assets affected include Member Demographics, Encounter Data, Claims Data, Enrollment Data, Premium Data, Risk Score, and Provider Demographics.

AWS SSO Credentials:
This asset includes the credentials used to sign into the AWS console. The impacts to confidentiality, integrity, and availability in the event of a breach could be as follows:
   C: An attacker gaining access to AWS SSO credentials and multi-factor authentication (MFA) could access data stored across the platform.
   I: An attacker gaining access to AWS SSO credentials and MFA could mutate data stored across the platform.
   A: An attacker gaining access to AWS SSO credentials and MFA could shut down infrastructure. This asset may be persisted on engineers' machines. The functional assets affected include all assets across the platform.

Stream API Credentials:
This asset includes the credentials used to connect to the chat system in each of the environments (development, staging, preview, production). The impacts to confidentiality, integrity, and availability in the event of a breach could be as follows:
   C: An attacker could access the chat logs of contract negotiation channels.
   I: An attacker could spoof themselves as a user and place harmful messages into the chat channel. This asset may be persisted in Vault and GitHub Secrets (Test Environment Only). The functional asset affected is the Contract Chat Channel.

Stream Dashboard Login:
This asset includes the credentials used to connect to dashboard.getstream.io. The impacts to confidentiality, integrity, and availability in the event of a breach could be as follows:
   C: An attacker could access the chat logs of contract negotiation channels and learn more about the system (user IDs, names) from data stored in Stream.
   I: An attacker could edit messages in channels via the “moderation” section.
   A: An attacker could remove “apps” and change API keys, causing a failure between the platform and Stream and shutting down chat. This asset may be persisted on engineers' machines. The functional asset affected is the Contract Chat Channel.

### ADDITIONAL CONSIDERATIONS

The present disclosure may be a system, a method, and/or a computer program product at any possible technical detail level of integration. The computer program product may include a computer readable storage medium (or media) having computer readable program instructions thereon for causing a processor to carry out aspects of the present disclosure. The computer readable storage medium can be a tangible device that can retain and store instructions for use by an instruction execution device. The computer readable storage medium may be, for example, but is not limited to, an electronic storage device, a magnetic storage device, an optical storage device, an electromagnetic storage device, a semiconductor storage device, or any suitable combination of the foregoing. A non-exhaustive list of more specific examples of the computer readable storage medium includes the following:
a portable computer diskette, a hard disk, a random access memory (RAM), a read-only memory (ROM), an erasable programmable read-only memory (EPROM or Flash memory), a static random access memory (SRAM), a portable compact disc read-only memory (CD-ROM), a digital versatile disk (DVD), a memory stick, a floppy disk, a mechanically encoded device such as punch-cards or raised structures in a groove having instructions recorded thereon, and any suitable combination of the foregoing. A computer readable storage medium, as used herein, is not to be construed as being transitory signals per se, such as radio waves or other freely propagating electromagnetic waves, electromagnetic waves propagating through a waveguide or other transmission media (e.g., light pulses passing through a fiber-optic cable), or electrical signals transmitted through a wire.

Computer readable program instructions described herein can be downloaded to respective computing/processing devices from a computer readable storage medium or to an external computer or external storage device via a network, for example, the Internet, a local area network, a wide area network and/or a wireless network. The network may comprise copper transmission cables, optical transmission fibers, wireless transmission, routers, firewalls, switches, gateway computers and/or edge servers. A network adapter card or network interface in each computing/processing device receives computer readable program instructions from the network and forwards the computer readable program instructions for storage in a computer readable storage medium within the respective computing/processing device.

Computer readable program instructions for carrying out operations of the present disclosure may be assembler instructions, instruction-set-architecture (ISA) instructions, machine instructions, machine dependent instructions, microcode, firmware instructions, state-setting data, configuration data for integrated circuitry, or either source code or object code written in any combination of one or more programming languages, including an object oriented programming language such as Smalltalk, C++, or the like, and procedural programming languages, such as the “C” programming language or similar programming languages. The computer readable program instructions may execute entirely on the user's computer, partly on the user's computer, as a stand-alone software package, partly on the user's computer and partly on a remote computer or entirely on the remote computer or server. In the latter scenario, the remote computer may be connected to the user's computer through any type of network, including a local area network (LAN) or a wide area network (WAN), or the connection may be made to an external computer (for example, through the Internet using an Internet Service Provider). In some aspects, electronic circuitry including, for example, programmable logic circuitry, field-programmable gate arrays (FPGA), or programmable logic arrays (PLA) may execute the computer readable program instructions by utilizing state information of the computer readable program instructions to personalize the electronic circuitry, in order to perform aspects of the present disclosure.

Aspects of the present disclosure are described herein with reference to flowchart illustrations and/or block diagrams of methods, apparatus (systems), and computer program products according to aspects of the disclosure. It will be understood that each block of the flowchart illustrations and/or block diagrams, and combinations of blocks in the flowchart illustrations and/or block diagrams, can be implemented by computer readable program instructions.

These computer readable program instructions may be provided to a processor of a general purpose computer, special purpose computer, or other programmable data processing apparatus to produce a machine, such that the instructions, which execute via the processor of the computer or other programmable data processing apparatus, create means for implementing the functions/acts specified in the flowchart and/or block diagram block or blocks. These computer readable program instructions may also be stored in a computer readable storage medium that can direct a computer, a programmable data processing apparatus, and/or other devices to function in a particular manner, such that the computer readable storage medium having instructions stored therein comprises an article of manufacture including instructions which implement aspects of the function/act specified in the flowchart and/or block diagram block or blocks.

The computer readable program instructions may also be loaded onto a computer, other programmable data processing apparatus, or other device to cause a series of operational steps to be performed on the computer, other programmable apparatus or other device to produce a computer implemented process, such that the instructions which execute on the computer, other programmable apparatus, or other device implement the functions/acts specified in the flowchart and/or block diagram block or blocks.

The flowchart and block diagrams in the Figures illustrate the architecture, functionality, and operation of possible implementations of systems, methods, and computer program products according to various aspects of the present disclosure. In this regard, each block in the flowchart or block diagrams may represent a module, segment, or portion of instructions, which comprises one or more executable instructions for implementing the specified logical function(s). In some alternative implementations, the functions noted in the blocks may occur out of the order noted in the Figures. For example, two blocks shown in succession may, in fact, be executed substantially concurrently, or the blocks may sometimes be executed in the reverse order, depending upon the functionality involved. It will also be noted that each block of the block diagrams and/or flowchart illustration, and combinations of blocks in the block diagrams and/or flowchart illustration, can be implemented by special purpose hardware based systems that perform the specified functions or acts or carry out combinations of special purpose hardware and computer instructions.

The terminology used herein is for the purpose of describing particular aspects only and is not intended to be limiting of the disclosure. As used herein, for example, the singular forms “a,” “an,” and “the” are intended to include the plural forms as well, unless the context clearly indicates otherwise. It will be further understood that the terms “contains”, “containing”, “includes”, “including,” “comprises”, and/or “comprising,” and variations thereof, when used in this specification, specify the presence of stated features, integers, steps, operations, elements, and/or components, but do not preclude the presence or addition of one or more other features, integers, steps, operations, elements, components, and/or groups thereof.

Terms of orientation used herein are merely for purposes of convention and referencing and are not to be construed as limiting. However, it is recognized these terms could be used with reference to an operator or user. Accordingly, no limitations are implied or to be inferred. In addition, the use of ordinal numbers (e.g., first, second, third, etc.) is for distinction and not counting. For example, the use of “third” does not imply there may be a corresponding “first” or “second.” Also, if used herein, the terms “coupled” or “coupled to” or “connected” or “connected to” or “attached” or “attached to” may indicate establishing either a direct or indirect connection, and is not limited to either unless expressly referenced as such. Furthermore, to the extent that the terms “includes,” “has,” “possesses,” and the like are used in the detailed description, claims, appendices and drawings such terms are intended to be inclusive in a manner similar to the term “comprising” as “comprising” is interpreted when employed as a transitional word in a claim. The term “based on” means “based at least in part on.” The terms “about” and “approximately” can be used to include any numerical value that can vary without changing the basic function of that value. When used with a range, “about” and “approximately” also disclose the range defined by the absolute values of the two endpoints, e.g. “about 2 to about 4” also discloses the range “from 2 to 4.” Generally, the terms “about” and “approximately” may refer to plus or minus 5-10 % of the indicated number.

While the disclosure has described several exemplary aspects, it will be understood by those skilled in the art that various changes can be made, and equivalents can be substituted for elements thereof, without departing from the spirit and scope of the disclosure. In addition, many modifications will be appreciated by those skilled in the art to adapt a particular instrument, situation, or material to aspects of the disclosure without departing from the essential scope thereof. Therefore, it is intended that the disclosure not be limited to the particular aspects disclosed, or to the best mode contemplated for carrying out this disclosure, but that the disclosure will include all aspects falling within the scope of the appended claims. Moreover, reference in the appended claims to an apparatus or system or a component of an apparatus or system being adapted to, arranged to, capable of, configured to, enabled to, operable to, or operative to perform a particular function encompasses that apparatus, system, or component, whether or not it or that particular function is activated, turned on, or unlocked, as long as that apparatus, system, or component is so adapted, arranged, capable, configured, enabled, operable, or operative.

What is claimed is:

1. A system for value-based care (VBC) contract construction, comprising:
a data manager implemented on a remote cloud service and configured to:
obtain, a first request from a client layer, the first request generated based on an action at a user device; and
construct, in response to the first request, at least one VBC value using at least one of a plurality of VBC tools; and
the client layer communicatively coupled to the data manager and configured to process the at least one VBC value to generate a user-interface for an implementation at the user device, the user-interface configured to enable a user to view VBC informatics based on the at least one VBC value on a display device and to make inputs to control visualization of the VBC informatics displayed to the user.

2. The system of claim 1, wherein the plurality of VBC tools includes an affinity tool configured to implement, while processing the at least one VBC value, at least one of a member identification, provider information, provider specialty information, affinity type information, a count of visits, visit span information, and last visit information.

3. The system of claim 1, wherein the plurality of VBC tools includes an attribution tool configured to implement, while processing the at least one VBC value, at least one of an attribution type, an attribution lookback, an attribution first choice, an attribution second choice, an attribution third choice, a tie breaker value, payer information, benefit plan information, provider specialty information, and exclusion criteria.

4. The system of claim 1, wherein the plurality of VBC tools includes a model type tool configured to implement, while processing the at least one VBC value, one of a medical loss ratio (MLR) model, a per member per month (PMPM) model, and a trend-based model.

5. The system of claim 1, wherein the plurality of VBC tools includes a cost terms tool configured to implement, while processing the at least one VBC value, baseline attribution information, a benchmark type, a baseline period, cost exclusions, performance attribution information, a performance period, and a performance cost.

6. The system of claim 1, wherein the plurality of VBC tools includes a quality parameters tool configured to implement, while processing the at least one VBC value, composite information, quality result application information, and quality performance incentive type.

7. The system of claim 1, wherein the plurality of VBC tools includes a choose a provider tool.

8. The system of claim 1, wherein the plurality of VBC tools includes a contract parameters tool configured to allow the user to perform itemized adjustments for one or more parameters, the itemized adjustments propagated to the at least one VBC value via the data manager.

9. The system of claim 8, wherein the one or more parameters comprise at least one of: administrative expense add-ons, claim corrections, incurred but not reported (IBNR) claim information, medical loss ratio (MLR) target information, and a shared savings/loss rate.

10. The system of claim 8, wherein the one or more parameters comprise at least one of: complete month information, administrative adjustment information, PMPM target information, and incurred but not reported (IBNR) claim information.

11. The system of claim 1, wherein the inputs are dynamic actuarial inputs configured to generate the at least one VBC value.

12. The system of claim 1, wherein the data manager is configured to facilitate implementing the plurality of VBC tools for multiple requests from a plurality of user devices.

13. A system for value-based care (VBC) contract construction, comprising:
a user device communicatively coupled to a client layer over a network and configured to output a first request to the client layer based on user inputs, the first request configured to cause at least one of a plurality of VBC tools to generate at least one VBC value; and
a user-interface displayed on the user device, the user-interface configured to enable a user to view VBC informatics based on the at least one VBC value displayed on the user device and to make the user inputs to control visualization of the VBC informatics displayed to the user.

14. The system of claim 13, wherein the plurality of VBC tools includes an affinity tool configured to implement, while processing the at least one VBC value, at least one of a member identification, provider information, provider specialty information, affinity type information, a count of visits, visit span information, and last visit information.

15. The system of claim 13, wherein the plurality of VBC tools includes an attribution tool configured to implement, while processing the at least one VBC value, at least one of an attribution type, an attribution lookback, an attribution first choice, an attribution second choice, an attribution third choice, a tie breaker value, payer information, benefit plan information, provider specialty information, and exclusion criteria.

16. The system of claim 13, wherein the plurality of VBC tools includes a model type tool configured to implement, while processing the at least one VBC value, one of a medical loss ratio (MLR) model, a per member per month (PMPM) model, and a trend-based model.

17. The system of claim 13, wherein the plurality of VBC tools includes a cost terms tool configured to implement, while processing the at least one VBC value, baseline attribution information, a benchmark type, a baseline period, cost exclusions, performance attribution information, a performance period, and a performance cost.

18. The system of claim 13, wherein the plurality of VBC tools includes a quality parameters tool configured to implement, while processing the at least one VBC value, composite information, quality result application information, and quality performance incentive type.

19. The system of claim 13, wherein the plurality of VBC tools includes a contract parameters tool configured to allow the user to perform itemized adjustments for one or more parameters, the itemized adjustments propagated to the at least one VBC value via a data manager.

20. The system of claim 13, wherein the user inputs are dynamic actuarial inputs configured to generate the at least one VBC value.
